(19) United States
(12) Patent Application Publication
(10) Pub. No.: US 2025/0234829 A1
(43) Pub. Date: Jul. 24, 2025

(54) METHODS AND COMPOSITIONS FOR MODIFYING FLOWERING TIME GENES IN PLANTS

(71) Applicant: Syngenta Crop Protection AG, Basel (CH)

(72) Inventors: Yang Gao, Beijing (CN); Chunxia Liu, Beijing (CN); Yuguo Zhang, Beijing (CN); Yanhui Chen, Beijing (CN); Dawei Liang, Beijing (CN); Xi Chen, Beijing (CN)

(73) Assignee: Syngenta Crop Protection AG, Basel (CH)

(21) Appl. No.: 18/845,952

(22) PCT Filed: Mar. 9, 2023
(86) PCT No.: PCT/US2023/064019

§ 371 Date: Sep. 11, 2024

(30) Foreign Application Priority Data
Mar. 11, 2022 (WO) PCT/CN2022/080339

(51) Int. Cl. A01H 6/54; A01H 1/02; A01H 1/04; C12N 9/22; C12N 15/113; C12N 15/82

(52) U.S. Cl. CPC A01H 6/542; A01H 1/021; A01H 1/04; C12N 9/22; C12N 15/113; C12N 15/827

(57) ABSTRACT

Methods and compositions are provided for modifying the flowering time and/or maturity time of soybean plants to enable them to be cultivated in a variety of geographical locations having different day lengths. Modified soybean plants are disclosed comprising non-natural mutant alleles at the E1 and/or E1Lb locus. The modified plants have a shorter flowering and/or maturity time than control plants.

### RELATED APPLICATION INFORMATION

This application claims priority to application PCT/CN2022/080339 filed Mar. 11, 2022, the contents of which are incorporated herein by reference in its entirety.

### STATEMENT REGARDING ELECTRONIC SUBMISSION OF A SEQUENCE LISTING

A Sequence Listing in ST.26 format, submitted under 37 C.F.R. § 1.821, entitled 82490.xml, 41 kb in size, generated on Mar. 9, 2023, and filed via EFS-Web is provided in lieu of a paper copy. This Sequence Listing is hereby incorporated by reference into the specification for its disclosures.

### FIELD

This disclosure relates to the field of plant biotechnology. In particular, it relates to methods and compositions for modifying the flowering time and/or maturity time of photoperiodic plants to enable them to be cultivated in a variety of geographical locations having different day lengths.

### BACKGROUND

Soybean (Glycine max) is a valuable field crop. Soybean oil extracted from the seed is employed in a number of retail products such as cooking oil, baked goods, margarines and the like. Soybean is also used as a grain as a food source for both animals and humans. Soybean meal is a component of many foods and animal feed. Production of edible protein ingredients from soybean offers a healthier and less expensive replacement for animal protein in meats as well as dairy-type products.

The typical growth cycle of full-season soybean begins with an extended period of vegetative growth. As shown at FIG. 1A, the vegetative stages begin with emergence of the hypocotyl out of the soil (VE) followed by unrolling of a pair of unifoliate leaves on the first node just above the cotyledons (VC). At this stage, the leaves are sufficiently unrolled so the leaf edges do not touch. Following VC, the plant goes from V1 (where fully developed leaves unfold at a first node) to Vn as trifoliate leaves unfold at each of a number of nodes (n).

The reproductive stages begin when a first open flower is present at any node on the main stem of the plant (R1 or beginning bloom). The first flower is typically towards the bottom of the plant. As the plant moves into full bloom, it enters into R2. At this stage, there is an open flower at one of the two uppermost nodes on the main stem with a fully developed flower. The reproductive stages include pod development (R3 and R4), seed development (R5 and R6), and finally maturity (R7 and R8). In R3, of beginning pod stage, there is a pod that is at least three-sixteenths-inch-long at one of the four uppermost nodes on the main stem with a fully developed leaf. In R4, or full pod, there is a at least three-quarter inch-long pod at one of the four uppermost nodes on the main stem with a fully developed leaf. In R5, or beginning seed stage, there is at least a one-eighth inch-long seed in a pod at one of the four uppermost nodes on the main stem with a fully developed leaf. In the R6 or full seed stage, there is a pod containing a green seed that fills the pod cavity at one of the four uppermost nodes on the main stem with a fully developed leaf. In the R7 or beginning maturity stage, there is at least one normal pod on the main stem that has reached its mature pod color. In the R8 or full maturity stage, at least 95 percent of the pods have reached their mature pod color. After R8, five to 10 days of drying weather are required to reduce soybean moisture levels to less than 15 percent.

Most flowering plants respond to daily photoperiodic cycles and are classified as either short day (SD) or long day (LD) plants based on the photoperiod conditions required to induce flowering. Photoperiod conditions experienced by a plant, in turn, are a function of the geographical location (e.g., latitude or longitude) where they are cultivated. Soybeans are short-day (SD) plants requiring days to be shorter than a critical value to induce flowering. Soybean varieties are classified into maturity groups according to their response to the photoperiod, such as based on the number of days till flowering occurs. For example, with a typical planting date of May 1st for most North American soy varieties, the vegetative period of soybean growth can last from 55-65 days with flowering beginning around mid-July.

Plant breeders and growers are always looking for new methods to manipulate the cultivation and yield of a plant, especially for agronomically important crops. For example, soybean breeders are interested in soybean varieties that can be cultivated in a wide range of geographical locations. Thus, there is a continuing need in the art for improved compositions and methods that modify agronomic traits related to flowering time.

Various genes have been identified and found to play roles in the control of flowering time and maturity of soybean. The present disclosure provides methods of creating novel, and non-natural, allelic variations of genes involved in soybean's photoperiodic response, and using specific allele combinations to provide soybean plants having a range of modified flowering times.

### SUMMARY

Methods and systems are provided for providing edited plants (e.g., soybean plants) having a flowering time and/or maturity time that is altered or modified from a natural flowering time (e.g., from a corresponding control plant that in unedited). In embodiments, the soybean plants have a flowering time and/or maturity time that is smaller (e.g., significantly smaller or slightly smaller) than the flowering time of the control plant. Compositions are also provided for nucleic acid molecules (e.g., expression cassettes or vectors) capable of introducing targeted edits in the genome of a plant cell, particularly in the locus of selected plant genes involved in regulating flowering and/or maturity and/or photoperiodic response, resulting in the creation of novel mutant alleles of the flowering and/or maturity and/or photoperiodic response genes. In embodiments, the resulting mutant alleles comprise sequences that when included in the genome a plant, confer the plants with a modified (e.g., smaller) flowering time and/or maturity time as compared to plants not comprising the mutant alleles. In embodiments, compositions resulting from different allelic combinations of the mutant alleles confer the plants with a modified (e.g., shorter/smaller) number of days for flowering, number of days for maturity, and/or number of days between flowering and maturity as compared to plants not comprising the allelic combination. Such compositions, and methods of using such compositions, enable plants to be produced that can be grown in a variety of geographical locations, including locations with shorter or longer days.

Embodiments of the invention include nucleic acid molecules comprising a nucleotide sequence encoding a mutation at an E1 locus and/or an E1LB locus of a genome of a soybean plant or plant cell, resulting in a novel allele at the E1 locus and/or E1LB locus. In embodiments, the mutation is introduced through an expression cassette comprising a nucleic acid sequence encoding the mutation at the E1 locus operably linked to a promoter and/or an E1LB locus operably linked to a promoter (e.g., same or different promoter). In embodiments, the mutation is introduced into the E1 locus and/or an E1LB locus of the genome of a soybean plant through genome editing (e.g., genome modification using a site directed nuclease. Embodiments of the invention include plants or plant cells comprising the novel alleles at the E1 locus and/or E1LB locus.

Further methods of the invention also include the use of mutagenesis and recombination (for example directed using chimeric oligonucleotides, Meganucleases, Zinc Fingers, TALEN or CRISPR) to introduce specific strand breaks, recombinational insertions and mutations so as to engineer in situ changes in plant genomes so that the thus mutated plant genome is then altered at the E1 and/or E1Lb loci. As a result, the plant is able to express one or more of a mutant E1 protein and a mutant E1Lb protein, and the plant is thus made to have an altered flowering time profile. Thus, the invention also includes altered flowering time plants, varieties and their seed and progeny that are derived from the product of application of the above methods of the invention.

Embodiments of the invention include example methods for establishing where a soybean plant, or seed thereof, should be grown. In example embodiments, the method comprises a) introducing, such as via genome modification using a site directed nuclease, a mutation at an E1 locus and/or an E1LB locus of a genome of a soybean plant; b) selfing the plant for one or more generations to generate a progeny plant that is homozygous at each of the E1 locus and the E1LB locus; c) obtaining DNA from said progeny plant; d) determining an allelic combination of said progeny plant via a first assay of the DNA indicative of a type of mutation introduced at the E1 locus and a second assay of the DNA indicative of a type of mutation introduced at the E1LB locus; and e) assigning a change in flowering time of the plant based on the determined allelic combination, wherein the change in flowering time is relative to a control plant not comprising the allelic combination. In embodiments, the editing of the endogenous E1 gene and E1LB gene comprises editing using a DNA modification enzyme (e.g., a site directed nuclease). In embodiments, the editing comprises introducing a mutation at a nuclear localization signal (NLS) at the E1 locus and the E1LB locus of the genome of the plant. In example embodiments, the editing comprises introducing a mutation at a basic domain of the NLS at the E1 locus and/or the E1LB locus (e.g, at both loci). In example embodiments, the editing comprises introducing a mutation at a second of two basic domains of the NLS at the E1 locus and the E1LB locus, wherein the second basic domain is downstream relative to a first basic domain of the NLS. In embodiments, the editing comprises transforming a plant cell with an expression cassette comprising (i) a nucleic acid that encodes the site-directed nuclease; and (ii) a nucleic acid that encodes at least one guide RNA (gRNA) directed to a target sequence. In embodiments, the at least gRNA is directed to a target sequence comprising a nuclear localization signal (NLS) at the E1 locus and the E1LB locus. In example embodiments, the at least one gRNA is directed to a target sequence comprising a second basic domain of the nuclear localization signal (NLS) at the E1 locus and the E1LB locus. In embodiments, the nucleic acid that encodes the site-directed nuclease is operably linked to a first promoter and the nucleic acid that encodes the at least one gRNA is operably linked to a second promoter. In embodiments, the expression cassette further comprises an enhancer operably linked to the first promoter or the second promoter. In embodiments, the site directed nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9-cytidine deaminase, chimeric Cas9-adenine deaminase, chimeric FEN1-Fokl, and Mega-TALs, a nickase Cas9 (nCas9), chimeric dCas9 non-Fokl nuclease and dCpf1 non-Fokl nuclease. In embodiments, the editing includes introducing into the E1 locus and the E1LB locus a mutation selected from the group consisting of an allele replacement, one or a plurality of base insertions, one or a plurality of base deletions.

In embodiments, the editing further comprises regenerating a transformed TO plant from the transformed plant cell, the transformed TO plant having a plurality of T1 seed, wherein the plurality of T1 seed contain a plurality of unique edits in the E1 locus and the E1LB locus; growing a plurality of T1 plants from the T1 seed; selfing the T1 plants for one or more generations to obtain a progeny plant that is homozygous at the E1 locus and the E1LB locus. In embodiments, the method further comprises sequencing the E1 locus and the E1LB locus of the progeny plant; sequencing the E1 locus and the E1LB locus of the TO plant; aligning the E1 locus and the E1LB locus sequence of the progeny plant with the corresponding sequence of the progeny plant to determine the type of mutation introduced into the E1 locus and the E1LB locus.

In embodiments, the allelic combination comprises: a loss of function E1 allele or a partial function E1 allele; and a loss of function E1LB allele or a partial function E1LB allele. In embodiments, assigning a change in flowering time comprises assigning a relative maturity group value relative to the control plant not comprising the allelic combination, wherein optionally the control plant comprises a wild-type allele at each of the E1 locus and E1LB locus. In embodiments, assigning a changing in flowering time comprises assigning a number of days by which the flowering time is advanced for the soybean plant relative to the control plant.

In other embodiments, a method of producing a soybean plant with a modified flowering time is provided. In example embodiments, the method comprises introducing an edit into an E1 gene of a plant cell to generate a mutant EL allele having reduced function of E1 protein relative to a wild-type E1 allele; introducing another edit in an E1LB gene of the plant cell to produce a mutant E1LB allele having reduced function of E1LB protein relative to a wild-type E1LB allele; and regenerating an edited plant from the edited plant cell; and selfing the edited plant to obtain an edited progeny comprising having allelic combination comprising the mutant E1 allele and the mutant E1LB allele, wherein the edited progeny has a flowering time that is modified relative to the flowering time of a control plant comprising the wild-type E1 allele and/or the wild-type E1LB allele. In embodiments, the method comprises introducing the edit into the E1 gene comprises introducing a plurality of base pair deletions into the E1 gene, wherein the mutant E1 allele encodes a mutated E1 protein comprising an in-frame deletion or a truncated E1 protein; and wherein introducing the edit into the E1LB gene comprises introducing a plurality of base pair deletions into the E1LB gene, wherein the mutant E1LB allele encodes a mutated E1LB protein having an in-frame deletion or a truncated E1LB protein. In embodiments, the truncated E1 protein has no functional activity relative to a wild-type E1 protein, the in-frame deletion mutated E1 protein has partial functional activity relative to the wild-type E1 protein, the truncated E1LB protein has no functional activity relative to a wild-type E1LB protein, and the in-frame deletion mutated E1LB protein has partial functional activity relative to the wild-type E1LB protein.

Embodiments of the invention further include gene edited plants and edited progeny plants comprising any of the recited allelic combinations. In embodiments, the flowering time of the edited progeny plant is modified (e.g., longer or shorter) than the flowering time of the control plant. In embodiments, a relative maturity group value of the edited progeny plant is different from the relative maturity group value of the control plant.

### Brief Description of the Sequences in the Sequence Listing

SEQ ID NO: 1 is the coding sequence (CDS) for a wild-type E1 gene from G. max.

SEQ ID NO: 2 is the amino acid (AA) sequence of the wild-type E1 protein encoded by SEQ ID NO: 1.

SEQ ID NO: 3 is the coding sequence (CDS) for a wild-type E1Lb gene from G. max.

SEQ ID NO: 4 is the amino acid (AA) sequence of the wild-type E1Lb protein encoded by SEQ ID NO: 3.

SEQ ID NO: 5 is the nucleotide sequence of a mutated E1 allele having an 8 bp deletion at positions 147 to 154 of SEQ ID NO: 1. The deletion results in an E1 allele having a loss of function.

SEQ ID NO: 6 is the amino acid (AA) sequence of a prematurely truncated E1 protein encoded by SEQ ID NO: 5. The truncated protein of SEQ ID NO: 6 does not display any E1 activity.

SEQ ID NO: 7 is the nucleotide sequence of a mutated E1 allele having a 13 bp deletion at positions 144 to 156 of SEQ ID NO: 1. The deletion results in an E1 allele having a loss of function. SEQ ID NO: 8 is the amino acid (AA) sequence of a prematurely truncated E1 protein encoded by SEQ ID NO: 7. The truncated protein of SEQ ID NO: 8 does not display any E1 activity.

SEQ ID NO: 9 is the nucleotide sequence of a mutated E1 allele having a 25 bp deletion at positions 131 to 155 of SEQ ID NO: 1. The deletion results in an E1 allele having a loss of function.

SEQ ID NO: 10 is the amino acid (AA) sequence of a prematurely truncated E1 protein encoded by SEQ ID NO: 9. The truncated protein of SEQ ID NO: 10 does not display any E1 activity.

SEQ ID NO: 11 is the nucleotide sequence of a mutated E1 allele having a 9 bp in-frame deletion at positions 146 to 154 of SEQ ID NO: 1. The deletion results in an E1 allele having partial functionality.

SEQ ID NO: 12 is the amino acid (AA) sequence of a mutated E1 protein having an in-frame deletion encoded by SEQ ID NO: 11. The in-frame deletion mutated E1 protein of SEQ ID NO: 12 displays reduced E1 activity.

SEQ ID NO: 13 is the nucleotide sequence of a mutated E1 allele having a 15 bp substitution at positions 145 to 159 of SEQ ID NO: 1. The resulting in-frame deletion results in an E1 allele having partial functionality.

SEQ ID NO: 14 is the amino acid (AA) sequence of a prematurely truncated E1 protein encoded by SEQ ID NO: 13. The in-frame deletion mutated E1 protein of SEQ ID NO: 14 does displays reduced E1 activity.

SEQ ID NO: 15 is the nucleotide sequence of a mutated E1Lb allele having a 7 bp deletion at positions 149 to 155 of SEQ ID NO: 3. The deletion results in an E1Lb allele having a loss of function.

SEQ ID NO: 16 is the amino acid (AA) sequence of a prematurely truncated E1Lb protein encoded by SEQ ID NO: 15. The truncated protein of SEQ ID NO: 16 does not display any E1Lb activity.

SEQ ID NO: 17 is the nucleotide sequence of a mutated E1Lb allele having a 139 bp deletion at positions 13 to 151 of SEQ ID NO: 3. The deletion results in an E1Lb allele having a loss of function.

SEQ ID NO: 18 is the amino acid (AA) sequence of a prematurely truncated E1Lb protein encoded by SEQ ID NO: 17. The truncated protein of SEQ ID NO: 18 does not display any E1Lb activity.

SEQ ID NO: 19 is the nucleotide sequence of a mutated E1Lb allele having a 3 bp in-frame deletion at positions 149 to 151 of SEQ ID NO: 3. The deletion results in an E1Lb allele having partial functionality.

SEQ ID NO: 20 is the amino acid (AA) sequence of a truncated E1Lb protein having an in-frame deletion encoded by SEQ ID NO: 19. The in-frame deletion mutated protein of SEQ ID NO: 20 displays reduced E1Lb activity.

SEQ ID NO: 21 is the nucleotide sequence of a mutated E1Lb allele having a 6 bp in-frame deletion at positions 149 to 154 of SEQ ID NO: 3. The deletion results in an E1Lb allele having partial functionality.

SEQ ID NO: 22 is the amino acid (AA) sequence of a mutated E1Lb protein having an in-frame deletion encoded by SEQ ID NO: 21. The in-frame deletion mutated protein of SEQ ID NO: 22 displays reduced E1Lb activity.

SEQ ID NO: 23 is the nucleotide sequence of a mutated E1Lb allele having a 9 bp in-frame deletion at positions 148 to 156 of SEQ ID NO: 3. The deletion results in an E1Lb allele having partial functionality.

SEQ ID NO: 24 is the amino acid (AA) sequence of a mutated E1Lb protein having an in-frame deletion encoded by SEQ ID NO: 22. The in-frame deletion mutated protein of SEQ ID NO: 22 displays reduced E1Lb activity.

SEQ ID NOS: 25-26 are the nucleotide sequences of a set of primers (forward and reverse primer, respectively) used during PCR to amplify an E1Lb allele.

SEQ ID NOS: 27-28 are the nucleotide sequences of a set of primers (forward and reverse primer, respectively) used during PCR to amplify an E1 allele.

SEQ ID NO: 29 is the nucleotide sequence of a primer used for sequencing an E1Lb allele.

SEQ ID NO: 30 is the nucleotide sequence of a primer used for sequencing an E1 allele.

SEQ ID NOS: 31-32 are the nucleotide sequences of a set of primers (forward and reverse primer, respectively) used during PCR to amplify an LbCas12a gene.

SEQ ID NOS: 33-34 are the nucleotide sequences of a set of primers (forward and reverse primer, respectively) used during PCR to amplify an Adh gene.

SEQ ID NO: 35 is the nucleotide sequence of a probe used for detecting LbCas12a.

SEQ ID NO: 36 is the nucleotide sequence of a probe used for detecting Adh.

SEQ ID NO: 37 is the nucleotide sequence of a synthetic guide RNA (gRNA) used to target the second basic domain of the nuclear localization signal (NLS) of both wild-type E1 and E1Lb proteins, thereby creating mutant E1 and E1Lb alleles.

### BRIEF DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1A shows the various developmental phases of a soybean plant.

FIG. 1B depicts variation in soy pod coloration upon maturity.

FIG. 2 is a schematic drawing of vector 25462 used for Agrobacterium-mediated transformation of soybean mature seeds to generate targeted mutations in GmE1 and GmE1Lb genes.

FIG. 3A shows the targeting of the gRNA (SEQ ID NO: 37) of vector 25462 (FIG. 2) to the second basic domain of NLS for both E1 and E1Lb genes.

FIG. 3B shows the alignment of the second basic domain of NLS for both E1 and E1Lb genes to determine a consensus sequence to be used for creating the gene editing gRNA target sequence.

FIGS. 4A-B shows the alignment of sequences of homozygous mutations at the E1 locus with a wild-type allele to determine the variation at the target sequence. Alignment at the nucleotide sequence level is shown at FIG. 4A. Alignment at the amino acid sequence level is shown at FIG. 4B.

FIGS. 5A-B shows the alignment of sequences of homozygous mutations at the E1Lb locus with a wild-type allele to determine the variation at the target sequence. Alignment at the nucleotide sequence level is shown at FIG. 5A. Alignment at the amino acid sequence level is shown at FIG. 5B.

FIG. 6 shows the alignment of sequences of in-frame deletion mutations at the second basic NLS domain of the target sequence, and the resulting in-frame deletion alleles, relative to a wild-type allele. Alignment is shown at the nucleotide sequence level and at the amino acid sequence level.

FIG. 7 shows the alignment of sequences of loss of function deletion mutations at the second basic NLS domain of the target sequence, and the resulting loss of function deletion alleles, relative to a wild-type allele. Alignment is shown at the nucleotide sequence level and at the amino acid sequence level.

FIG. 8 shows the phenotype of E1 generation soybean plants comprising novel allelic combinations.

FIGS. 9-11 show the phenotype of E2 generation soybean plants comprising novel allelic combinations.

FIG. 12 shows the phenotype of E2 generation soybean plants comprising novel allelic combinations.

FIG. 13 shows the phenotype of E3 generation soybean plants comprising novel allelic combinations.

### DEFINITIONS

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “allele” refers to a variant or an alternative nucleotide sequence of a gene or at a particular genetic locus. Such an allele can be considered (i) wild-type or (ii) mutant if one or more mutations or edits are present in the nucleic acid sequence of the mutant allele relative to the wild-type allele. In diploids, a single allele is inherited by a progeny individual separately from each parent at each locus.

The two alleles of a given locus present in a diploid organism occupy corresponding places on a pair of homologous chromosomes, although one of ordinary skill in the art understands that the alleles in any particular individual do not necessarily represent all of the alleles that are present in the species.

A mutant allele for a gene may have a reduced or eliminated activity or expression level for the gene relative to the wild-type allele. For diploid organisms such as corn and soy, a first allele can occur on one chromosome, and a second allele can occur at the same locus on a second homologous chromosome. If one allele at a locus on one chromosome of a plant is a mutant allele and the other corresponding allele on the homologous chromosome of the plant is wild type, then the plant is described as being heterozygous for the mutant allele. However, if both alleles at a locus are mutant alleles, then the plant is described as being homozygous for the mutant alleles. A plant homozygous for mutant alleles at a locus may comprise the same mutant allele or different mutant alleles if heteroallelic or biallelic.

“Allelic variation” refers to the phenomenon of variation in the sequence form of an allele at a given genetic locus. Allelic variation results in the creation of two or more allelic variants. The variants may be naturally occurring and reflective of genetic differences among individuals of the same species. Such natural variations can occur as a result of natural breeding patterns. Alternatively, the variants may be non-naturally occurring, and artificially created (e.g., by a breeder or a scientist), such as using mutagenesis and/or gene editing techniques. In embodiments of the invention, allelic variants of the soybean E1 gene and/or E1LB gene are created through gene editing methods that result in the introduction of a mutation. In additional or alternative embodiments of the invention, allelic variants of the soybean E1 gene and/or E1LB gene may be created through chemical mutagenesis, transposon insertion or excision, or any other known mutagenesis technique.

In example embodiments, the mutation introduced into the E1 and/or E1LB locus is an allele replacement, one or a plurality of base pair insertions, or one or a plurality of base pair deletions. The base pair insertions or base pair deletions may include a 3n base mutation wherein a multiple of 3 base pairs are deleted or inserted (e.g., insertion or deletion of 3 bp, 6 bp, 9 bp, 12 bp, 15 bp, 18 bp, etc.), thereby not affecting the reading frame of the gene. Alternatively, the base pair insertion or deletion may not be a multiple of 3 base pairs (e.g., an insertion or deletion of 2 bp, 4 bp, 5 bp, 7 bp, 11 bp, etc.), thereby affecting the reading frame of the gene.

In particular embodiments, the mutation is a truncation mutation wherein the mutation can result in the introduction of a stop codon into the gene at a location earlier than intended. Transcription of the resulting mutant allele is terminated at the earlier than intended stop codon, resulting in a truncated protein that is shorter than the corresponding wild-type protein.

In other particular embodiments, the mutation is an in-frame deletion mutation wherein deletion of an integral multiple of three base pairs (that is, 3n base pairs) occurs. Since three base pairs encode a single amino acid, the result of the in-frame deletion is that the reading frame of the transcript is maintained (that is, no frameshift mutations are introduced), however, the transcript generated from the mutant allele encodes a mutated protein that is shorter than the corresponding wild-type protein. Both the in-frame deletion and truncation mutations in the gene result in a mutated allele that encodes a shortened protein having reduced function (e.g., partial loss of function or complete loss of function) compared to the protein encoded by the wild-type (i.e., unmutated) allele.

As used herein, an “allelic combination” refers to the specific combination of alleles present at more than one characterized location or loci. Embodiments of the invention include a plurality of allelic combinations at the E1 and E1LB loci. Non-limiting examples of allelic combinations at the E1 and E1LB loci include:
(i) a first allelic combination comprising a mutant E1LB allele resulting in partial expression of E1LB protein and a wild-type E1 allele;
(ii) a second allelic combination comprising a mutant E1LB allele resulting in loss of expression of E1LB protein and a wild-type E1 allele;
(iii) a third allelic combination comprising a mutant E1LB allele resulting in partial expression of E1LB protein and a mutant E1 allele resulting in partial expression of E1 protein;
(iv) a fourth allelic combination comprising a mutant E1LB allele resulting in loss of expression of E1LB protein and a mutant E1 allele resulting in partial expression of E1 protein;
(v) a fifth allelic combination comprising a mutant E1LB allele resulting in partial expression of E1LB protein and a mutant E1 allele resulting in loss of expression of E1 protein;
(vi) a sixth allelic combination comprising a mutant E1LB allele resulting in loss of expression of E1LB protein and a mutant E1 allele resulting in loss of expression of E1 protein;
(vii) a seventh allelic combination comprising a wild-type E1LB allele and a mutant E1 allele resulting in partial expression of E1 protein;
(viii) an eighth allelic combination comprising a wild-type E1LB allele and a mutant E1 allele resulting in loss of expression of E1 protein; and
(ix) a ninth allelic combination comprising wild-type alleles at both the E1 and E1LB loci.

In embodiments, the flowering time of a non-naturally occurring soybean plant comprising any of the first to eighth non-naturally occurring allelic combinations disclosed above is modified relative to a control plant comprising the ninth allelic combination of the wild-type E1 allele and the wild-type E1LB allele. In particular embodiments, the modified flowering time of a non-naturally occurring soybean plant comprising any of the first to eighth non-naturally occurring allelic combinations is smaller (e.g., slightly smaller or significantly smaller) than the flowering time of the ninth allelic combination of the wild-type E1 allele and the wild-type E1LB allele.

Based on the resulting phenotype, the mutant alleles at a locus may be dominant or recessive. In embodiments, the first allelic combination comprising a mutant E1LB allele resulting in partial expression of E1LB protein and a wild-type E1 allele is homozygous recessive at the E1LB locus and homozygous dominant at the E1locus; the second allelic combination comprising a mutant E1LB allele resulting in loss of expression of E1LB protein and a wild-type E1 allele is homozygous recessive at the E1LB locus and homozygous dominant at the E1locus; the third allelic combination comprising a mutant E1LB allele resulting in partial expression of E1LB protein and a mutant E1 allele resulting in partial expression of E1 protein is homozygous recessive at the E1LB locus and homozygous recessive at the E1locus; the fourth allelic combination comprising a mutant E1LB allele resulting in loss of expression of E1LB protein and a mutant E1 allele resulting in partial expression of E1 protein is homozygous recessive at the E1LB locus and homozygous recessive at the E1locus; the fifth allelic combination comprising a mutant E1LB allele resulting in partial expression of E1LB protein and a mutant E1 allele resulting in loss of expression of E1 protein is homozygous recessive at the E1LB locus and homozygous recessive at the E1locus; the sixth allelic combination comprising a mutant E1LB allele resulting in loss of expression of E1LB protein and a mutant E1 allele resulting in loss of expression of E1 protein is homozygous recessive at the E1LB locus and homozygous recessive at the E1locus; the seventh allelic combination comprising a wild-type E1LB allele and a mutant E1 allele resulting in partial expression of E1 protein is homozygous dominant at the E1LB locus and homozygous recessive at the E1locus; the eighth allelic combination comprising a wild-type E1LB allele and a mutant E1 allele resulting in loss of expression of E1 protein is homozygous dominant at the E1LB locus and homozygous recessive at the E1locus; and the ninth allelic combination comprising wild-type alleles at both the E1 and E1LB loci is homozygous dominant at the E1LB locus and homozygous dominant at the E1locus.

In embodiments of the invention, the allelic combination of a plant at the E1 and E1LB loci may be determined via molecular marker-based assays, such as a first assay of the DNA of the plant indicative of a type of mutation introduced at the E1 locus and a second assay of the DNA indicative of a type of mutation introduced at the E1LB locus.

In embodiments, the allelic combination is indicative of a change in flowering time of the plant relative to a control plant not comprising the allelic combination (e.g., a control plant comprising one or more of the wild-type alleles or comprising the ninth allelic combination of wild type alleles at both loci).

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

As used herein, the phrase “associated with” refers to a recognizable and/or assayable relationship between two entities. For example, the phrase “associated with soybean maturity” refers to a trait, locus, gene, allele, marker, phenotype, etc., or the expression thereof, the presence or absence of which can influence a number of days a soybean plant spends in a vegetative state. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with an allele for soybean maturity gene E1” refers to a marker whose presence or absence can be used to predict whether an E1 allele is present and responsible for the flowering time of the plant.

A “dominant maturity allele” is an allele that, when present either in single copy (heterozygous) or two copies (homozygous), affects the maturity of the plant. A “recessive maturity allele” is an allele that affects the maturity of the plant only when present in two copies (homozygous), and does not affect the maturity of a plant when present in a single copy (heterozygous).

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole. As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising,” “consisting essentially of,” and “consisting of,” where one of these three terms is used herein, the presently disclosed and claimed subject matter can include in some embodiments the use of either of the other two terms. For example, if a subject matter relates in some embodiments to soybean plants that comprise in their genome a genomic interval comprising a mutant E1LB allele, it is understood that the disclosed subject matter thus also encompasses soybean plants with genomic intervals that in some embodiments consist essentially of the mutant E1LB allele as well as soybean plants with genomic intervals that in some embodiments consist of the mutant E1LB allele. Similarly, it is also understood that in some embodiments the methods for the disclosed subject matter comprise the steps that are disclosed herein, in some embodiments the methods for the presently disclosed subject matter consist essentially of the steps that are disclosed, and in some embodiments the methods for the presently disclosed subject matter consist of the steps that are disclosed herein.

As used herein, a “cultivar” is a race or variety of a plant that has been created or selected intentionally and maintained through cultivation.

As used herein, “determinate growth habit” refers to ceasing of vegetative growth after the main stem terminates in a cluster of flowers. In comparison, “indeterminate growth habit” refers to the development of leaves and flowers simultaneously throughout a portion of their reproductive period, with one to three pods at the terminal apex.

As used herein, the “E1 gene” or an allele thereof, is in reference to GmE1 or Glyma.06G207800 (www(.)soybase(.)org) associated with flowering time and maturity and located at the pericentromeric region of chromosome 6. The E1 gene (SEQ ID NO:1) is intron-free and encodes an E1 protein (SEQ ID NO: 2) that contains a putative bipartite nuclear localization signal (NLS) and a domain distantly related to the plant-specific B3 domain (B3-like domain) (Xia, Z. J. et al. Proc Natl Acad Sci USA. 109, E2155-E2164 (2012)). E1 is a transcription factor. E1 is considered to be a contributor to the variation in flowering time among soybean cultivars. E1 also has an impact on pre-flowering development in addition to post-flowering response. E1 is expressed in a bimodal pattern, with higher expression in long-day (LD) conditions than in short-day (SD) conditions. E1 is a putative transcription factor (TF) that negatively controls GmFT2a and GmFT5a to delay flowering under the background with functional PHYA genes (E3, E4) and LD conditions.

Mutations to the E1 gene, natural or deliberately introduced, result in altered E1 activity and a modified flowering time in soybean plants comprising the mutated gene. In embodiments of the present invention, mutations to the E1 gene result in non-natural E1 alleles having reduced activity than the corresponding wild-type alleles. Modified plants comprising the non-natural E1 alleles have a modified flowering time and/or maturity time than plants comprising wild-type E1 alleles. In particular embodiments, the modified plants comprising the non-natural E1 alleles have a smaller or shorter flowering time than the plants comprising the wild-type E1 alleles.

As used herein, the “E1LB gene” or an allele thereof, is in reference to the soybean gene GmE1Lb or Glyma.04G143300.1 (www(.)soybase(.)org), on chromosome 4 of the soybean genome, that can control the onset of flowering (Xu, M. et al. Plant Physiol. 168, 1735-1746 (2015)). This gene functions similarly to E1 in flowering. Virus-induced silencing of E1Lb was found to up-regulate the expression of FT2a and FT5a and lead to early flowering. E1Lb retards flowering under long-day conditions by repressing the expression of FT2a and FT5a independently of E1. Mutations to the E1Lb gene, natural or deliberately introduced, result in altered E1Lb activity and a modified flowering time in soybean plants comprising the mutated gene. In embodiments of the present invention, mutations to the E1Lb gene result in non-natural E1 alleles having reduced activity than the corresponding wild-type alleles. Modified plants comprising the non-natural E1Lb alleles have a modified flowering time and/or maturity time than plants comprising wild-type E1Lb alleles. In particular embodiments, the modified plants comprising the non-natural E1Lb alleles have a smaller or shorter flowering time than the plants comprising the wild-type E1Lb alleles.

As used herein, the term “gene” refers to a hereditary unit including a sequence of DNA that occupies a specific location on a chromosome and that contains the genetic instruction for a particular characteristic or trait in an organism.

A “genetic map” is a description of genetic linkage relationships among loci on one or more chromosomes within a given species, generally depicted in a diagrammatic or tabular form. The genetic map is distinct from a physical map which is a description of the location of a genetic element (e.g., gene, allele, chromosomal locus, marker, etc.) on a sequenced chromosome.

As used herein, the term “human-induced mutation” refers to any mutation that occurs as a result of either direct or indirect human action. This term includes, but is not limited to, mutations obtained by any method of targeted mutagenesis and gene editing.

As used herein, “introduced” means delivered, expressed, applied, transported, transferred, permeated, or other like term to indicate the delivery, whether of nucleic acid or protein or combination thereof, of a desired object to an object. For example, nucleic acids encoding a site directed nuclease and optionally at least one guide RNA may be introduced into a plant embryo. Likewise, extant editing machinery (comprising a site directed nuclease protein and optionally at least one guide RNA) may be introduced into sterilized soybean seeds upon application of appropriate cell-penetrating chemicals and/or peptides.

As used herein, “line” refers to a group of individual plants from the similar parentage with similar traits. An “elite line” is any line that has resulted from breeding and selection for superior agronomic performance. Additionally, an elite line is sufficiently homogenous and homozygous to be used for commercial production. Elite lines may be used in the further breeding efforts to develop new elite lines. An elite plant is any plant from an elite line.

As used herein, “locus” is a chromosomal locus or region where a polymorphic nucleic acid, trait determinant, gene, or marker is located. A “locus” can be shared by two homologous chromosomes to refer to their corresponding locus or region.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

As used herein, the term “molecular marker” can be used to refer to a genetic marker, as defined above, or an encoded product thereof (e.g., a protein) used as a point of reference when identifying the presence/absence of a gene or allele (such as an allele at a soybean maturity associated locus, such as at an E1 and/or E1LB locus). A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence. As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, “modified” in the context of a plant, plant seed, plant part, plant cell, and/or plant genome, refers to a plant, plant seed, plant part, plant cell, and/or plant genome comprising an engineered change in the expression level and/or coding sequence of one or more of an E1 gene and an E1LB gene relative to a wild-type or control plant, plant seed, plant part, plant cell, and/or plant genome, such as via a genome editing event or mutation affecting (e.g., reducing or eliminating) the expression level or activity of one or more endogenous E1 and/or E1LB genes. The term “modified” may further refer to a plant, plant seed, plant part, plant cell, and/or plant genome having one or more mutations affecting expression of one or more endogenous E1 and/or E1LB genes introduced through chemical mutagenesis, transposon insertion or excision, or any other known mutagenesis technique, or introduced through genome editing. For clarity, therefore, a modified plant, plant seed, plant part, plant cell, and/or plant genome includes a mutated and/or edited plant, plant seed, plant part, plant cell, and/or plant genome having a modified expression level, expression pattern, and/or coding sequence of one or more of an E1 and E1LB gene(s) relative to a wild-type or control plant, plant seed, plant part, plant cell, and/or plant genome. Modified plants may be homozygous or heterozygous for any given mutation or edit, and/or may be bi-allelic at the E1 and/or E1LB gene locus. A modified plant is bi-allelic for the E1 and/or E1LB gene if each copy of the gene is modified by a different allele (i.e., different mutation(s) and/or edit(s)), wherein each allele lowers the expression level and/or activity of the gene. Modified plants or seeds may contain various molecular changes that affect expression of E1 and/or E1LB gene(s), including genetic and/or epigenetic modifications. Modified plants, plant parts, seeds, etc., may have been subjected to mutagenesis, genome editing or site-directed integration (e.g., without being limiting, via methods using site-specific nucleases), genetic transformation (e.g., without being limiting, via methods of Agrobacterium transformation or microprojectile bombardment), or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the E1 and/or E1LB gene. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

As used herein, the term “nucleotide sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif, United States of America. See also Smith & Waterman, 1981; Needleman & Wunsch, 1970; Pearson & Lipman, 1988; Ausubel et al., 1988; and Sambrook & Russell, 2001.

The term “open reading frame” (ORF) refers to a nucleic acid sequence that encodes a polypeptide. In some embodiments, an ORF comprises a translation initiation codon (i.e., start codon), a translation termination (i.e., stop codon), and the nucleic acid sequence there between that encodes the amino acids present in the polypeptide. The terms “initiation codon” and “termination codon” refer to a unit of three adjacent nucleotides (i.e., a codon) in a coding sequence that specifies initiation and chain termination, respectively, of protein synthesis (mRNA translation).

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In other cases, a phenotype is the result of interactions among several genes, which in some embodiments also results from an interaction of the plant and/or plant cell with its environment.

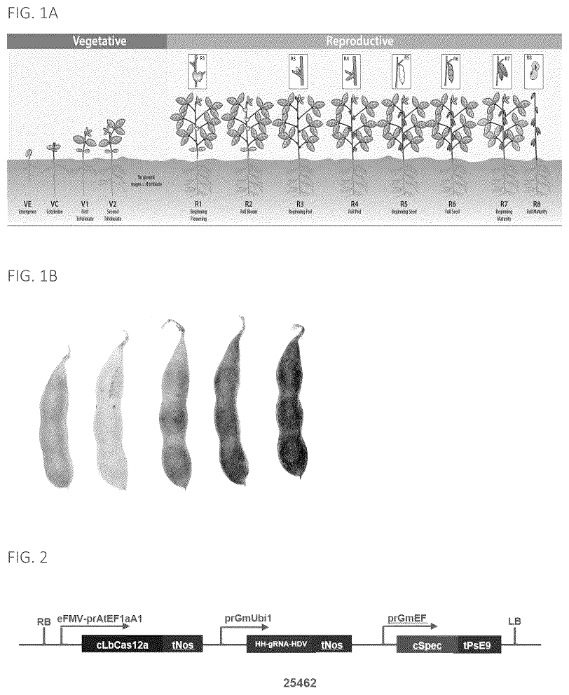

In the case of soybean maturity genes such as E1 and E1LB, the phenotypic trait includes one or more or a combination of flowering time, post-flowering time, relative maturity, maturity time, maturity group and number of days from flowering of the soybean plant to beginning of maturity. In particular embodiments, the phenotypic trait measured is a flowering time and includes a measure of time elapsed between the VE and R1 phase of a modified soybean plant (see FIG. 1A for the various stages) relative to a control plant. In another embodiment, the phenotypic trait measured is a maturity time and includes a measure of time elapsed between the R1 and R7 phase, or R1 and R8 phase, of the modified soybean plant (see FIG. 1A for the various stages) relative to a control plant. The number of days may vary, relative to a control plant comprising wild-type alleles at both loci, based on the specific allelic combination of the plant.

In example embodiments of the invention, a modified soybean plant comprising a mutant E1 allele and a wild-type E1LB allele has a flowering time and/or maturity time that is significantly smaller than that of the control plant, while a soybean plant comprising a mutant E1LB allele and a wild-type E1 allele has a flowering time and/or maturity time that is slightly smaller than that of the control plant. Further, a soybean plant comprising a mutant allele at both E1 and E1LB loci has a flowering time and/or maturity time that is significantly smaller than that of the control plant. A duration or degree by which the flowering time is reduced is further based on the nature of the mutant allele, such as based on whether the mutated allele results in a gain of function, complete loss of function, or partial loss of function of the gene product. Furthermore, the duration or degree by which the flowering time is reduced may be based on whether the allele is homozygous or heterozygous (e.g., bi-allelic), and whether the allele is homozygous recessive or dominant.

As used herein, a modified plant having a flowering time and/or maturity time that is “slightly smaller” than a control plant has a flowering time and/or maturity time that is between 1 and 10 days shorter than the control plant (e.g., shorter than that of the control plant by at least 1 day, 2 days, 3 days, 4 days, 5 days, 6 days, 7 days, 8 days, 9 days or 10 days). In embodiments, the flowering time of a modified plant is slightly smaller than the control plant if the flowering time is shorter by 1-2 days, 1-3 days, 1-4 days, 1-5 days, 1-6 days, 1-7 days, 1-8 days, 1-9 days or 1-10 days.

In comparison, a modified plant having a flowering time and/or maturity time that is “significantly smaller” than the control plant has a flowering time and/or maturity time that is at least 10 days shorter than that of the control plant, such as between 10-100 days shorter than the control plant (e.g., shorter than that of the control plant by at least 10 days, 10-20 days 10-30 days, 10-40 days, 10-50 days, 10-60 days, 10-70 days, 10-80 days, 10-90 days or 10-100 days or any range therebetween such as 20-30 days, 20-40 days, 30-40 days, 40-50 days, 50-60 days, 70-80 days, 80-90 days, 90-100 days, and so on).

As used herein, the term “photoperiodic response” or “photoperiodism” refers to the physiological reaction of a plant to the relative lengths of light and dark periods. Photoperiod responsive plants may be “short-day”, “long-day” or “day-neutral” plants. Photoperiodism affects flowering by inducing the shoot to produce floral buds instead of leaves and lateral buds. Soybean, for example, is a short-day (SD) plant. In embodiments of the invention, soybean flowering time is assessed. Short day plants flower when the night lengths exceed their critical photoperiod and cannot flower under short nights. They require a continuous period of darkness before floral development can begin. Natural nighttime light, such as moonlight or lightning, is not of sufficient brightness or duration to interrupt flowering. Typically, short-day (i.e. long-night) plants flower as days grow shorter (e.g., late summer and fall in the northern hemisphere). The length of the dark period required to induce flowering differs among species and varieties of a species. Long-day plants flower when the night length falls below their critical photoperiod. These plants typically flower as days get longer (e.g., late spring and early summer in the northern hemisphere).

As used herein, “flowering time” or “days to flowering” is an estimate of a duration (e.g., in terms of hours, days, weeks, etc.) elapsed between initiation of first flowering and seed emergence. In embodiments of the invention, flowering time of a soybean plant is modified or altered, relative to a control plant, through the introduction of novel non-naturally occurring alleles in genes involved in soybean maturity, particularly E1 and/or E1Lb genes. In particular embodiments, flowering time is defined as a number of days elapsed for a soybean plant to transition from a VE stage (e.g., seeds emergence wherein cotyledons have been pulled through the soil surface for at least 50% of the seeds) to an R1 stage (e.g., beginning of flowering wherein at least 50% of the plants have at least one flower on any node). In particular embodiments, maturity time or post flowering time is defined as a number of days elapsed for a soybean plant to transition from the R1 stage (e.g., beginning of bloom wherein there is one open flower at any node on the main stem) to an R7 stage (wherein any pod has reached a mature pod color) or from the R1 stage to an R8 stage (wherein 95% of the pods have reached their mature pod color). A description of the various development stages of a soybean plant and mature soy pod coloration is provided at FIGS. 1A and 1B as reference.

As used herein, “relative maturity group” or “relative maturity value” or “RM” can be any indicative number, symbol, or combination of both, that provides an indication of when a plant will mature. In embodiments, the relative maturity value is indicative of an average number of days that elapse between flowering time and maturity time or between flowering and at last seed pod reaching maturity (e.g., to the R7 stage). Similarly, a change in relative maturity group may be associated with a change in the average flowering time. As an example, in North America, a change in relative maturity of one, from RM 2.0 to RM 3.0 may correlate with a change in maturity time of 10 days. This value may vary based on growing conditions, such as based on whether the plant was grown under short day or long day conditions or based on whether the plant was grown in greenhouse or field conditions.

As used herein, “altered” or “modified” means increased or decreased at maturity. In this aspect, a mature seed as defined by a seed that is harvested in the field for commercial agricultural practices, such as sale for feed. In an aspect, a soybean plants are selected for preferred geographies for expression of at least one phenotypic trait. The phenotypic trait includes altered levels of a substance or a molecule, such as proteins, oils, or gamma linolenic acid. “Altered” can include any relative increase or decrease of function or production of a gene product of interest, in an aspect up to and including complete elimination of function or production of that gene product. When levels of a gene product are compared, such a comparison is preferably carried out between organisms with a similar genetic background. Preferably, a similar genetic background is a background where the organisms being compared share 50% or greater, more preferably 75% or greater, and, even more preferably 90% or greater sequence identity of nuclear genetic material. In another aspect, a similar genetic background is a background where the plants are isogenic except for one or more markers of the present invention.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the terms “progeny” and “progeny plant” refer to a plant generated from vegetative or sexual reproduction from one or more parent plants. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1s, F2s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids.

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer in some embodiments to a meiotic crossover.

As used herein, the term “reference sequence” refers to a defined nucleotide sequence used as a basis for nucleotide sequence comparison.

As used herein, the term “reference plant” or “control plant” refers to a defined plant used as a basis for genetic and/or phenotypic comparison. In some embodiments, a soybean plant comprising wild-type alleles at each of the E1 and E1LB loci is a control plant for comparing to other plants comprising mutant alleles at one or both of the E1 and E1LB loci.

As used herein, the term “regenerate,” and grammatical variants thereof, refers to the production of a plant from tissue culture.

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances. Longer sequences typically hybridize specifically at higher temperatures. An extensive guide to the hybridization of nucleic acids is found in Sambrook & Russell, 2001. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Exemplary stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. Additional exemplary stringent hybridization conditions include 50% formamide, 5×SSC, and 1% SDS incubating at 42° C.; or SSC, 1% SDS, incubating at 65° C.; with one or more washes in 0.2×SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al., 1999).

As used herein, the term “trait” refers to a phenotype of interest, a gene that contributes to a phenotype of interest, as well as a nucleic acid sequence associated with a gene that contributes to a phenotype of interest. For example, a “soybean maturity trait” refers to a phenotype as well as a gene (e.g., E1 or E1LB) that contributes to soybean maturity and has a nucleic acid sequence (e.g., a soybean maturity-associated gene product) that is associated with a photoperiod response, including a number of days between flowering and pod formation.

As used herein, the term “transgene” refers to a nucleic acid molecule introduced into an organism or one or more of its ancestors by some form of artificial transfer technique. The artificial transfer technique thus creates a “transgenic organism” or a “transgenic cell.” It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

### DETAILED DESCRIPTION

Soybean is a short day (SD) plant grown in a wide range of geographical regions and over a wide range of latitudes from equatorial to up to 50 degrees. This wide adaptability has most likely been created by genetic diversity at a large number of the major genes and quantitative trait loci controlling flowering behavior.

Photoperiod is one of the leading climatic factors in determining soybean floral development and adaptation to different regions. Short day lengths can hasten flowering, whereas long day lengths can delay flowering. Due to their photoperiodic sensitivity, the cultivation area of each soybean cultivar is restricted to a very narrow range of latitudes to attain its highest yield.

Provided herein are plants comprising non-naturally occurring allelic combinations of soybean maturity genes, particularly at E1 and E1LB loci, that modify the flowering profile (e.g., flowering time, and/or maturity value) of the resulting plant. In some instances, the allelic combination results in a modified soybean maturity profile when the alleles are expressed in a plant or part thereof as compared to a control plant that does not comprise the given allelic combination, such as a control plant comprising wild-type alleles at one or both loci. As a result of the modified flowering profile, the resulting soybean plants, and their progeny plants, can be cultivated and grown in a wider range of latitudes, enabling higher yields. The terms “soybean maturity” and “relative maturity” and “photoperiod response” are used interchangeably herein. Various means of introducing mutations that result in the non-naturally occurring alleles and allelic combinations into the soybean plant are also disclosed, which include transgenic means, gene editing, and breeding. Markers for identifying the presence of these non-naturally occurring alleles in the plant are also disclosed. As used herein, the terms “phenotype,” “phenotypic trait” or “trait” refer to a distinguishable characteristic(s) of a genetically controlled trait.

In some embodiments, the plants provided herein are a non-naturally occurring variety of soybean having the desired trait. In specific embodiments, the non-naturally occurring variety of soybean is an elite soybean variety. A “non-naturally occurring variety of soybean” is any variety of soybean that does not naturally exist in nature. A “non-naturally occurring variety of soybean” may be produced by any method known in the art, including, but not limited to, transforming a soybean plant or germplasm, transfecting a soybean plant or germplasm, and crossing a naturally occurring variety of soybean with a non-naturally occurring variety of soybean. In some embodiments, a “non-naturally occurring variety of soybean” may comprise one of more heterologous nucleotide sequences. In some embodiments, a “non-naturally occurring variety of soybean” may comprise one or more non-naturally occurring alleles of a naturally occurring gene (i.e., non-naturally occurring mutations introduced into a gene that naturally occurs in soybean). In some embodiments, a “non-naturally occurring variety of soybean” may comprise a non-natural combination of one or more non-naturally alleles of soybean maturity gene (i.e., non-naturally occurring alleles of E1 and E1LB genes in different combinations that do not naturally occur in the same soybean).

A “subject plant or plant cell” is one in which genetic alteration, such as a mutation, has been affected as to a gene of interest to create a non-naturally occurring and novel allele, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has a wild-type allele at both the E1 and E1LB loci; (c) a plant or plant cell of the same genotype as the starting material but which has a wild-type allele at either the E1 or the E1LB locus; (d) a plant or plant cell which is a non-transformed segregant among progeny of a subject plant or plant cell; (e) a plant or plant cell genetically identical to the subject plant or plant cell but which is not exposed to conditions or stimuli that would induce expression of the gene or allele of interest; or (f) the subject plant or plant cell itself, under conditions in which the gene or allele of interest is not expressed.

I. Novel, Non-Naturally Occurring E1 Gene Alleles that Result in a Modified Soybean Flowering Time

Methods and compositions are provided for producing soybean plants having a modified flowering profile. Such plants comprise non-naturally occurring alleles at one or both of an E1 locus and an E1LB locus. These plants can be grown in geographic regions, including latitudes, that are outside of the region they would have been limited to if the allelic combination was not introduced (e.g., relative to a plant comprising any both of the wild-type alleles). In addition to widening the range of cultivation, the yield of the plant is also increased.

Site-directed mutations at the E1 and E1Lb loci are followed by selection of alleles encoding mutant forms of the E1 and E1Lb proteins that have reduced activity. Such alleles are useful for making soybean plants that have an altered flowering time. Novel E1 and E1Lb alleles thus created are especially suitable in the context of in situ-mutated (genome-edited) plants that can be grown in latitudes outside of their unmutated counterparts.

Non-limiting examples of allelic combinations resulting from the non-naturally occurring alleles described herein include:
(i) a mutant E1LB allele resulting in partial expression of E1LB protein and a wild-type E1 allele;
(ii) a mutant E1LB allele resulting in loss of expression of E1LB protein and a wild-type E1 allele;
(iii) a mutant E1LB allele resulting in partial expression of E1LB protein and a mutant E1 allele resulting in partial expression of E1 protein;
(iv) a mutant E1LB allele resulting in loss of expression of E1LB protein and a mutant E1 allele resulting in partial expression of E1 protein;
(v) a mutant E1LB allele resulting in partial expression of E1LB protein and a mutant E1 allele resulting in loss of expression of E1 protein; and
(vi) a mutant E1LB allele resulting in loss of expression of E1LB protein and a mutant E1 allele resulting in loss of expression of E1 protein.
(vii) a mutant E1 allele resulting in partial expression of E1 protein and a wild-type E1LB allele;
(viii) a mutant E1 allele resulting in loss of expression of E1 protein and a wild-type E1 allele.
The flowering time of a soybean plant comprising any of the above-mentioned allelic combinations is modified relative to a control plant. In particular embodiments, the control plant comprises a wild-type E1 allele and a wild-type E1LB allele.

### (a) E1 Gene and Novel Alleles Thereof

A large number of QTLs in different linkage groups have been identified to be involved in flowering and maturity. Among these, 11 have been confirmed as major effect QTLs that control the time to flowering and maturity in soybean: E1 and E2, E3, E4, E6, E7, E8, E9, E10, qDTF-J1 and J. The genes underlying QTLs E1-E4, E9, E10, qDTF-J1, and J have been identified, and their functions in the photoperiod control of flowering have been characterized. Among these genes, E1-E4 are four major loci associated with flowering time (FT) and maturity.

E1 is a major gene associated with flowering time and maturity and is located at the pericentromeric region. The E1 gene (SEQ ID NO:1) is intron-free and encodes an E1 protein (SEQ ID NO: 2) that contains a putative bipartite nuclear localization signal (NLS) and a domain distantly related to the plant-specific B3 domain (B3-like domain) (Xia, Z. J. et al. Proc Natl Acad Sci USA. 109, E2155-E2164 (2012)). E1 is a transcription factor. E1 is considered to be a contributor to the variation in flowering time among soybean cultivars. E1 also has an impact on pre-flowering development in addition to post-flowering response.

E1 gene includes at least 4 allelic natural variations with E1 and e1-as as the two basic genotypes. Known natural E1 recessive allele variations in soybean varieties include e1-as, e1-fs, e1-nl, and e1-b3a (Zhai, H. et al. Plos One. 9(5), e97636 (2014)). e1-as includes a single missense point mutation at the region of the nuclear localization signal that results in a leaky allele. This one amino acid change led to the cell localization change and e1 protein distribution in the nucleus and cytoplasm at the same time. However, e1-as is a leaky allele and has partially function of delaying flowering (Xia, 2013).

The other three nonfunctional alleles are e1-fs, e1-nl and e1-b3a. e1-fs has 1 bp deletion in the B3 domain, and this frameshift mutation resulted in a premature stop codon and a truncated protein encoding 41 amino acids. e1-nl is a null allele and all the E1 gene is deleted. e1-b3a allele has 3 SNPs and 2 bp deletions in the B3 domain resulting in frameshift mutation. E1-nl is a null allele with an ˜130kb deletion comprising the E1 gene. e1-b3a comprises a 2 bp deletion in the middle of the B3 domain. e1-as is apparently a leaky allele and partially suppresses flowering in soybean contrary to the functional e1-fs and e1-nl.

E1 is expressed in a bimodal pattern, with higher expression in long-day (LD) conditions than in short-day (SD) conditions. E1 is a putative transcription factor (TF) that negatively controls GmFT2a and GmFT5a to delay flowering under the background with functional PHYA genes (E3, E4) and LD conditions (Lin, X. et al. Molecular mechanisms for the photoperiodic regulation of flowering in soybean. J. Integr. Plant Biol. (2021) 63: 981-994; Xia, Z. et al. Positional cloning and characterization reveal the molecular basis for soybean maturity locus E1 that regulates photoperiodic flowering. Proc. Natl. Acad. Sci. USA (2021) 109: E2155-2164; Zhai, H. et al. Diurnal expression pattern, allelic variation, and association analysis reveal functional features of the E1 gene in control of photoperiodic flowering in soybean. PloS One (2015) 10 (8), e0135909. doi: 10.1371/journal.pone.0135909).

As used herein, E1 activity refers to the effect of the E1 protein on flowering time and/or maturity time. Alleles resulting in a complete loss of function encode a mutated E1 protein having no E1 activity. Alleles resulting in a partial loss of function encode a mutated E1 protein having reduced E1 activity relative to the wild type, unmutated E1 protein.

The present invention discloses novel E1 alleles created using mutagenesis and genome editing techniques. The novel alleles are created using deletion and/or substitution of bases at positions identified corresponding to the wild-type allele. Example alleles comprising non-naturally occurring mutations in the E1 gene are provided at SEQ ID NOS: 5, 7, 9, 11, and 13, encoding corresponding mutated E1 proteins provided at SEQ ID NOS: 6, 8, 10, 12, and 14. At least some of the non-naturally occurring alleles (e.g., SEQ ID NOS: 5, 7, and 9) result in a mutated E1 protein have no E1 activity. At least some of the non-naturally occurring alleles (e.g., SEQ ID NOS: 11 and 13) result in a mutated E1 protein have reduced or limited E1 activity.

### (b) E1Lb Gene and Novel Alleles Thereof

Similar to E1, two E1 orthologs, namely, E1La (Glyma.04G156400.1) and E1Lb (Glyma.04G143300.1), can control the onset of flowering. (Xu, M. et al. Plant Physiol. 168, 1735-1746 (2015)). These genes function similarly to E1 in flowering Virus-induced silencing of E1La and E1Lb was found to up-regulate the expression of FT2a and FT5a and lead to early flowering. Natural loss-of-function allele (E1Lb) exhibited upregulated expression of FT2a and FT5a, and flowered earlier than those for E1Lb under long-day conditions in both the e3/E4 and E3/E4 genetic backgrounds. E1Lb retards flowering under long-day conditions by repressing the expression of FT2a and FT5a independently of E1.

As used herein, E1Lb activity refers to the effect of the E1Lb protein on flowering time and/or maturity time. Alleles resulting in a complete loss of function encode a mutated E1Lb protein having no E1Lb activity. Alleles resulting in a partial loss of function encode a mutated E1Lb protein having reduced E1Lb activity relative to the wild-type, unmutated E1 protein.

The present invention discloses novel E1Lb alleles created using mutagenesis and genome editing techniques. The novel alleles are created using deletion and/or substitution of bases at positions identified corresponding to the wild-type allele.

Example alleles comprising non-naturally occurring mutations in the E1Lb gene are provided at SEQ ID NOS: 15, 17, 19, 21, and 23, encoding corresponding mutated E1Lb proteins provided at SEQ ID NOS: 16, 18, 20, 22, and 24. At least some of the non-naturally occurring alleles (e.g., SEQ ID NOS: 15 and 17) result in a mutated E1Lb protein have no E1Lb activity. At least some of the non-naturally occurring alleles (e.g., SEQ ID NOS: 19, 21, and 23) result in a mutated E1Lb protein have reduced or limited E1 activity.

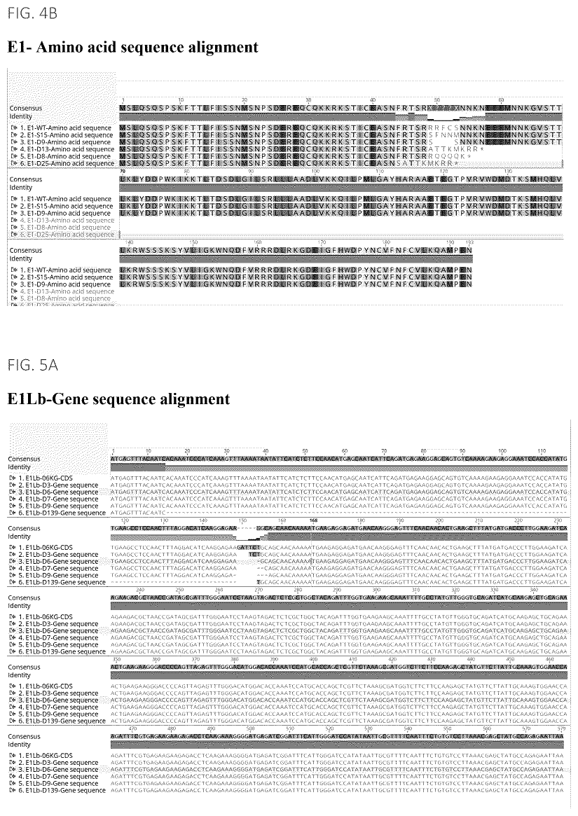

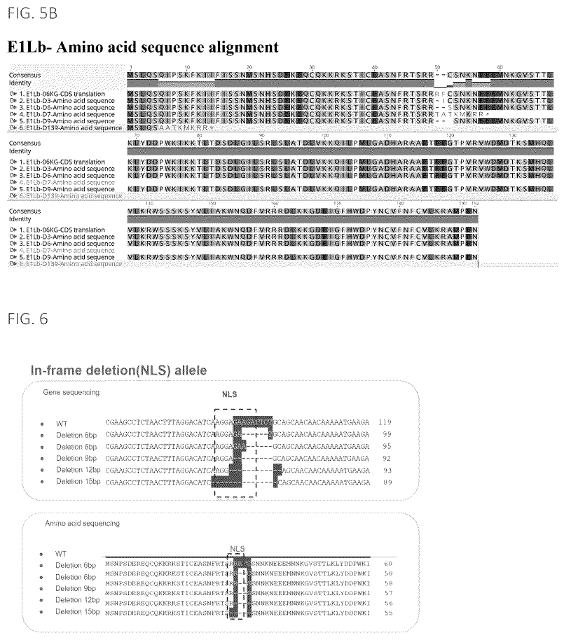

The term “corresponding to” in the context of nucleic acid sequences or means that when the nucleic acid sequences of certain sequences are aligned with each other, the nucleic acids that “correspond to” certain enumerated positions in the present invention are those that align with these positions in a reference sequence, but that are not necessarily in these exact numerical positions relative to a particular nucleic acid sequence of the invention. For example, an E1 allele generated via a mutation comprising an 8 bp deletion at nucleotide 147 to 154 corresponds means that when the nucleotide sequence of the wild-type allele is aligned with the nucleotide sequence of the mutant allele, there is an eight base pair gap in the mutant allele which overlaps with nucleotide 147 to 154 of the wild-type allele. In other words, the mutant allele overlaps with the wild-type allele at positions before, and not including position 147, as well overlapping at positions after, and not including position 154. An example alignment of a wild-type allele and the corresponding novel alleles is shown at FIGS. 4A and 5A.

Optimal alignment of sequences for comparison can be conducted by computerized implementations of known algorithms. or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

In some embodiments, the alleles result in variants and fragments of the above-described E1 and E1Lb proteins and the variants and fragments result in a modified flowering time profile when expressed in a plant, plant part, or seed.

Alleles that result in fragments of the E1 or E1Lb proteins that modify flowering time profile when expressed in a plant, plant part, or seed include those that are shorter than the full-length sequences, either due to the use of an alternate downstream start site, or due to processing that produces a shorter protein having the activity. An allele encoding a fragment of a protein that modifies flowering time profile when expressed in a plant can be a polypeptide that is, for example, 10, 25, 50, 100, 150, 200, 250 or more amino acids in length of any one of SEQ ID NOS: 2 or 4. As used herein, a fragment comprises at least 8 contiguous amino acids of SEQ ID NO: 2 or 4. Unless otherwise stated, identity and similarity will be calculated by the Needleman-Wunsch global alignment and scoring algorithms (Needleman and Wunsch (1970) J. Mol. Biol. 48(3):443-453) as implemented by the “needle” program, distributed as part of the EMBOSS software package (Rice, P., Longden, I., and Bleasby, A., EMBOSS: The European Molecular Biology Open Software Suite, 2000, Trends in Genetics 16, (6) pp276-277, versions 6.3.1 available from EMBnet at embnet.org/resource/emboss and emboss.sourceforge.net, among other sources) using default gap penalties and scoring matrices (EBLOSUM62 for protein and EDNAFULL for DNA). Equivalent programs may also be used. By “equivalent program” is intended any sequence comparison program that, for any two sequences in question, generates an alignment having identical nucleotide residue matches and an identical percent sequence identity when compared to the corresponding alignment generated by needle from EMBOSS version 6.3.1.

Additional mathematical algorithms are known in the art and can be utilized for the comparison of two sequences. See, for example, the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLAST programs of Altschul et al. (1990) J. Mol. Biol. 215:403. BLAST nucleotide searches can be performed with the BLASTN program (nucleotide query searched against nucleotide sequences) to obtain nucleotide sequences homologous to nucleic acid molecules of the invention, or with the BLASTX program (translated nucleotide query searched against protein sequences) to obtain protein sequences homologous to nucleic acid molecules of the invention. BLAST protein searches can be performed with the BLASTP program (protein query searched against protein sequences) to obtain amino acid sequences homologous to protein molecules of the invention, or with the TBLASTN program (protein query searched against translated nucleotide sequences) to obtain nucleotide sequences homologous to protein molecules of the invention. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Hemkoff et al. (1992) Proc. Natl. Acad. Sci. USA 89: 10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

### II. Gene Editing

The flowering time associated sequences provided herein, that is E1 and E1LB, can be targeted within the genome of a recipient plant cell to create the novel allele sequences. Such methods include, but are not limited to, meganucleases designed against the plant genomic sequence of interest CRISPR-Cas9, TALENs, and other technologies for precise editing of genomes (Feng, et al. Cell Research 23: 1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701: 147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol: 51-65); prime editing and transposases (Anzalone, A. et al., Nat Biotechnol. 2020 July; 38(7):824-844); translocation; and inversion.

Various embodiments of the methods described herein use gene editing. In some embodiments, gene editing is used to mutagenize the genome of a plant to produce plants having novel E1 alleles and/or novel E1Lb alleles that confer a modified flowering time. The novel alleles may be created by targeted introduction of mutations in the genome of a plant at the E1 and/or E1Lb loci. In particular embodiments, the editing comprises introducing a mutation (e.g., insertion, deletion, frameshift, etc.) at a nuclear localization signal (NLS) at the E1 locus and the E1LB locus. In the present study, a point mutation from arginine to threonine at position 15 in E1 occurs at exactly the first basic domain of KKRK (which changes to KKTK) in the bipartite NLS, leading to different subcellular distributions than either E1 and e1-as. This leads the inventors to believe that the second basic domain may have a similar role in E1 protein distribution. In particular embodiments, the editing comprises introducing a mutation (e.g., insertion, deletion, frameshift, etc.) at a basic domain of the NLS at the E1 locus and/or the E1LB locus. In particular embodiments, the editing comprises introducing a mutation at a second of two basic domains of the NLS at the E1 locus and the E1LB locus, wherein the second basic domain is downstream relative to a first basic domain of the NLS.

Editing may be achieved through the use of editing expression cassettes. The expression cassette will include in the 5′-3′ direction of transcription, a transcriptional and translational initiation region (i.e., a promoter), a polynucleotide of interest, and a transcriptional and translational termination region (i.e., termination region) functional in the organism of interest, i.e., a plant or bacteria. The promoters of the invention are capable of directing or driving transcription and expression of a coding sequence in a host cell. The regulatory regions (i.e., promoters, transcriptional regulatory regions, and translational termination regions) may be endogenous or heterologous to the host cell or to each other. As used herein, a chimeric gene or a chimeric nucleic acid molecule comprises a coding sequence operably linked to a transcription initiation region that is heterologous to the coding sequence.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV pSOY1 terminator, the tml terminator, the nopaline synthase terminator and the pea rbcs E9 terminator. These can be used in both monocotyledons and dicotyledons. In addition, a gene's native transcription terminator may be used. Termination regions used in the expression cassettes can be obtained from, e.g., the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (990) Plant Cell 2: 1261-1272; Munroe et al. (1990) Gene 91: 151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al, eds. (1980).

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, tissue-preferred, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference. In some embodiments, the promoter used herein comprises an exogenous promoter. The term “exogenous promoter,” refers to a promoter that is not found in plants in nature, for example, a synthetic promoter.

In some embodiments, provided herein are plants transformed with and expressing gene-editing machinery as described above, which, when crossed with a target plant, result in gene editing in the target plant.

In general, gene editing may involve transient, inducible, or constitutive expression of the gene editing components or systems. Gene editing may involve genomic integration or episomal presence of the gene editing components or systems.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer to the plant an improved trait, e.g., modified flowering time profile. The recombination template can be introduced into the plant to be edited either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising an allele of interest, e.g., SEQ ID NOS: 1, 2 or a polynucleotide encoding a polypeptide having the sequence of any one of SEQ ID NOS: 2, 4 may be provided on/as a template. By designing the system such that one or more single strand or double strand breaks are introduced within, upstream, and/or downstream of the corresponding region in the genome of a plant not comprising the gene sequence of interest, this region can be replaced with the template.

In embodiments, the site directed nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9-cytidine deaminase, chimeric Cas9-adenine deaminase, chimeric FEN1-Fokl, and Mega-TALs, a nickase Cas9 (nCas9), chimeric dCas9 non-Fokl nuclease and dCpf1 non-Fokl nuclease.

In embodiments, the editing is performed by transforming a plant cell with an expression cassette comprising (i) a nucleic acid that encodes the site-directed nuclease; and (ii) a nucleic acid that encodes at least one guide RNA (gRNA) directed to a target sequence. The gRNA is directed to a target sequence which comprises the nuclear localization sequence (NLS) at the E1 locus and/or the E1LB locus. In particular embodiments, the gRNA is directed to a target sequence comprising the second basic domain of the nuclear localization sequence (NLS) at the E1 locus and/or the E1LB locus. In embodiments of the editing expression cassette, the nucleic acid that encodes the site-directed nuclease is operably linked to a first promoter while the nucleic acid that encodes the at least one gRNA is operably linked to a second promoter, which may be the same or different from the first promoter. In some embodiments, the expression cassette may further comprise one or more additional regulatory elements, such as an enhancer operably linked to the first promoter or the second promoter.

In some embodiments, mutations in the genes or wild-type alleles of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type V1 CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide. In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a“spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Gene editing methods and compositions are also disclosed in U.S. Pat. No. 10,519,456 and 10,285,348 82, the entire content of which is herein incorporated by reference. The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference.

In another aspect, provided herein is a method of editing plant genomic DNA. In some embodiments, the method comprises using a first soybean plant expressing a DNA modification enzyme and at least one optional guide nucleic acid as described above to pollinate a target plant comprising genomic DNA to be edited. In some embodiments, the method of creating novel alleles and allelic combinations comprises, editing, via a site directed nuclease, at one or more of an E1 locus and/or an E1LB locus of a genome of a soybean plant.

III. Selection of Plants with Novel Alleles and Improved Traits.

In addition to the phenotypic traits, the genetic characteristic of the plant as represented by its genetic marker profile can be used to select plants of desired traits. The term “marker-based selection” refers to the use of genetic markers to detect one or more nucleic acids from the plant, where the nucleic acid is associated with a desired trait to identify plants that carry genes or alleles for desirable (or undesirable) traits. Markers include but are not limited to Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as Microsatellites, and Single Nucleotide Polymorphisms (SNPs). There are known sets of public markers that are being examined by ASTA and other industry groups for their applicability in standardizing determinations of what constitutes an essentially derived variety under the US Plant Variety Protection Act. However, these standard markers do not limit the type of marker and marker profile which can be employed in breeding or developing backcross conversions, or in distinguishing varieties or plant parts or plant cells or verify a progeny pedigree. Primers and PCR protocols for assaying these and other markers are disclosed in the Soybase (sponsored by the USDA Agricultural Research Service and Iowa State University) located at the world wide web at 129.186.26.94/SSR.html.

The term “associated with” as used herein refers to a recognizable and/or detectable relationship between two entities. For example, the phrase “associated with modified flowering time” refers to a trait, locus, gene, allele, marker, phenotype, etc., or the expression product thereof, the presence or absence of which can influence or indicate an extent and/or degree to which a plant or its progeny exhibits a change in its flowering time and/or maturity value as compared to a control plant. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence (or absence) of the marker is an indicator of whether the allele is present (or absent) in a plant, germplasm, or population comprising the marker. For example, “a marker associated with a novel E1 allele that confers a modified flowering time profile” refers to a marker whose presence or absence can be used to determine whether a novel E1 allele is present in a plant, and/or to what extent the plant will display an alteration in the flowering time as compared to a control plant. Likewise, “a marker associated with a novel E1Lb allele that confers a modified flowering time profile” refers to a marker whose presence or absence can be used to determine whether a novel E1Lb allele is present in a plant, and/or to what extent the plant will display an alteration in the flowering time as compared to a control plant.

The term “allele(s)” refer to any of one or more alternative forms of a gene, all of which alleles relate to at least one trait or characteristic. In a diploid cell, the two alleles of a given gene occupy corresponding loci on a pair of homologous chromosomes.

The term “genotype” and variants thereof refer to the genetic composition of an organism, including, for example, whether a diploid organism is heterozygous (i.e., has two different alleles for a given gene or QTL) or homozygous (i.e., has the same allele for a given gene or QTL) for one or more genes or loci (e.g., a SNP, a haplotype, a gene mutation, an insertion, or a deletion).

In one embodiment, the markers used to identify the plants comprising the alleles disclosed herein are SNPs. Non-limiting examples of SNP genotyping methods include hybridization, primer extension, oligonucleotide ligation, nuclease cleavage, minisequencing and coded spheres. Such methods are well known and disclosed in e.g., Gut, I. G., Hum. Mutat. 17: 475-492 (2001); Shi, Clin. Chem. 47(2): 164-172 (2001); Kwok, Pharmacogenomics 1(1): 95-100 (2000); and Bhattramakki and Rafalski, Discovery and application of single nucleotide polymorphism markers in plants, in PLANT GENOTYPING: THE DNA FINGERPRINTING OF PLANTS, CABI Publishing, Wallingford (2001). A wide range of commercially available technologies utilize these and other methods to interrogate SNPs, including Masscode SupTM/Sup (Qiagen, Germantown, MD, (Hologic, Madison, WI), (Applied Biosystems, Foster City, CA), (Applied Biosystems, Foster City, CA) and Beadarrays SupTM/Sup (Illumina, San Diego, CA).

In some embodiments, an assay (e.g., generally a two-step allelic discrimination assay or similar), a KASP SupTM/Sup assay (generally a one-step allelic discrimination assay defined below or similar), or both can be employed to identify the SNPs that associate with modified flowering time profile. In an exemplary two-step assay, a forward primer, a reverse primer, and two assay probes that recognize two different alleles at the SNP site (or hybridization oligos) are employed. The forward and reverse primers are employed to amplify genetic loci that comprise SNPs that are associated with modified FT profile. The particular nucleotides that are present at the SNP positions are then assayed using the probes. In some embodiments, the assay probes and the reaction conditions are designed such that an assay probe will only hybridize to the reverse complement of a 100% perfectly matched sequence, thereby permitting identification of which allele (s) that are present based upon detection of hybridizations. In some embodiments, the probes are differentially labeled with, for example, fluorophores to permit distinguishing between the two assay probes in a single reaction. Exemplary methods of amplifying include employing a polymerase chain reaction (PCR) or ligase chain reaction (LCR) using a nucleic acid isolated from a soybean plant or germplasm as a template in the PCR or LCR.

In some embodiments, a number of SNP alleles together within a sequence, or across linked sequences, can be used to describe a haplotype for any particular genotype. Ching et al., BMC Genet. 3: 19 (2002) (14 pages); Gupta et al., (2001) Curr Sci. 80:524-535, Rafalski, Plant Sci. 162: 329-333 (2002). In some cases, haplotypes can be more informative than single SNPs and can be more descriptive of any particular genotype. For example, a single SNP may be allele “T” for a specific disease resistant line or variety, but the allele “T” might also occur in the soybean breeding population being utilized for recurrent parents. In this case, a combination of alleles at linked SNPs may be more informative. Once a unique haplotype has been assigned to a donor chromosomal region, that haplotype can be used in that population or any subset thereof to determine whether an individual has a particular gene. The use of automated high throughput marker detection platforms known to those of ordinary skill in the art makes this process highly efficient and effective.

These SNP markers can be used in a marker assisted breeding program to move traits, such as native traits or traits conferred by transgenes or traits conferred by genome editing, into a desired plant background. As used herein, the term “native trait” refers to a trait already existing in germplasm, including wild relatives of crop species, or that can be produced by recombination of existing traits. For example, progeny plants from a cross between a donor soybean plant comprising in its genome a nucleic acid sequence encoding the alleles of SEQ ID NOS: 5-14, and a recipient soybean plant not comprising said nucleic acid sequence can be screened to detect the presence of the markers associated with modified FT profile. Plants comprising said markers can be selected and verified for modified FT profile as compared to control plants. In some embodiments, the markers comprise the primer sequences disclosed and described in Example 3.

### IV. Plant Transformation

Once the gene editing cassette has been cloned into an expression system, it is transformed into a plant cell. The receptor and target expression cassettes of the present invention can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

By “stably introducing” or “stably introduced” in the context of a polynucleotide introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations. Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra Gene 19: 259-268 (1982); Bevan et al., Nature 304:184-187 (1983)), the pat and bar genes, which confer resistance to the herbicide glufosinate (also called phosphinothricin; see White et al., Nucl. Acids Res 18: 1062 (1990), Spencer et al. Theor. Appl. Genet 79: 625-631 (1990) and U.S. Pat. Nos. 5,561,236 and 5,276,268), the hph gene, which confers resistance to the antibiotic hygromycin (Blochinger & Diggelmann, Mol. Cell Biol. 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), the glyphosate N-acetyltransferase (GAT) gene, which also confers resistance to glyphosate (Castle et al. (2004) Science, 304:1151-1154; U.S. Patent App. Pub. Nos. 20070004912, 20050246798, and 20050060767); and the mannose-6-phosphate isomerase gene, which provides the ability to metabolize mannose (U.S. Pat. Nos. 5,767,378 and 5,994,629).

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens.

These typically carry at least one T-DNA border sequence and include vectors such as pBIN19 (Bevan, Nucl. Acids Res. (1984)). For the construction of vectors useful in Agrobacterium transformation, see, for example, US Patent Application Publication No. 2006/0260011, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. For the construction of such vectors, see, for example, US Application No. 20060260011, herein incorporated by reference.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Preferred techniques include direct gene transfer into protoplasts using PEG or electroporation techniques, and particle bombardment into callus tissue.

Transformations can be undertaken with a single DNA species or multiple DNA species (i.e. co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

The genetic properties engineered into the genome-edited or transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the genome-edited or transgenic plants and seeds according to the invention can further be made in plant breeding.

Depending on the desired properties, different breeding measures are taken. The relevant techniques are well known in the art and include but are not limited to hybridization, inbreeding, backcross breeding, multi-line breeding, variety blend, interspecific hybridization, aneuploid techniques, etc. Thus, the genome edited or transgenic seeds and plants according to the invention can be used for the breeding of improved plant lines that, for example, increase the geographical range of cultivation. Many suitable methods for transformation using suitable selection markers such as kanamycin, binary vectors such as from Agrobacterium and plant regeneration as, for example, from tobacco leaf discs are well known in the art.

### V. Establishment of Cultivation Region Based on Altered Flowering Time Profile.

Methods are provided for establishing where a plant having genome edits in one or more of the E1 and E1Lb loci should be cultivated. Particularly, the methods enable establishment of where a soybean plant, or seed thereof, should be grown, wherein the soybean plant has genome edits at one or both of the E1 and E1Lb loci, resulting in a novel allelic combination, different from the allelic combination of a control plant comprising wild-type versions of the alleles. The method requires determination of the specific allelic combination followed by comparison of the altered flowering time resulting from the specific allelic combination. In example embodiments, assigning a changing in flowering time comprises assigning a duration, such as a number of hours, days, or weeks, by which the flowering time is changed (e.g, advanced or retarded) for the soybean plant relative to the control plant. For example, an allelic combination that results in a flowering time (e.g., duration from VE to R1) of 50 days, as compared to a control plant with a flowering time of 45 days is assigned a change (particularly, advancement) in flowering time of (50-45) 5 days or (5×24) 120 hours. In some embodiments an advancement of 5 days is associated with a defined relative maturity group shift. In other embodiments, an advancement of 10 days is associated with a defined relative maturity group shift. However, it will be appreciated that the relation between degree and direction of change in flowering time and change in relative maturity group may not be linear and may be based on additional factors such as ambient weather conditions (e.g., temperature and humidity), cultivation location (indoors or outdoors), watering and chemical treatment frequency, etc.

As a first non-limiting example, a change in flowering time from a first time of ˜90-100 days to a second time of ˜87-97 (e.g., 87 to 95) days may be correlated with a change in maturity group from RM 5.5 to RM 5.

As a second non-limiting example, a change in flowering time from a first time of ˜90-100 days to a second time of ˜83-84 days may be correlated with a change in maturity group from RM 5.5 to RM 4.

As a third non-limiting example, a change in flowering time from a first time of ˜90-100 days to a second time of ˜70-72 days may be correlated with a change in maturity group from RM 5.5 to RM 3.

As a fourth non-limiting example, a change in flowering time from a first time of ˜90-100 days to a second time of ˜58-62 days may be correlated with a change in maturity group from RM 5.5 to RM 2.

As a fifth non-limiting example, a change in flowering time from a first time of ˜90-100 days to a second time of ˜43-44 days may be correlated with a change in maturity group from RM 5.5 to RM 0 or RM 1.0.

As a sixth non-limiting example, a change in flowering time from a first time of ˜90-100 days to a second time of ˜37-41 days may be correlated with a change in maturity group from RM 5.5 to RM 00.

As a seventh non-limiting example, a change in flowering time from a first time of ˜90-100 days to a second time of ˜30-31 days may be correlated with a change in maturity group from RM 5.5 to RM 000.

In embodiments, the altered flowering time can be used to establish an altered maturity value, or relative maturity group of the edited plant. Based on the change in flowering time, a geographic region (e.g., latitude, temperature region, etc.) may be selected. In embodiments, selecting a region may further comprise determining whether to grow the plant indoors (e.g., in a greenhouse) or outdoors (e.g., in fields). As an example, based on the allelic combination, a soybean plant may be transitioned from growth in a first region to a second region. As used herein, transitioning the growth of the plant from a first region to a second region means that the edited plant can additionally or optionally be grown in the second region while the unedited control plant, from which the edited plant is derived or relative to which the change in flowering time is measured, continues to be grown in the first region only, and wherein the control plant cannot be grown in the second region.

As an example, based on the allelic combination, an edited non-naturally occurring soybean plant may be transitioned from growth in a first region between 40° N and 50° N (e.g., between 40° N and 45° N, or between 45° N and 50° N) to a second region between 30° N and 40° N (e.g., between 30° N and 35° N, or between 35° N and 40° N), or between 20° N and 30° N (e.g., between 20° N and 25° N, or between 25° N and 30° N). As another example, based on the allelic combination, an edited non-naturally occurring soybean plant may be transitioned from growth in a first region between 30° N and 40° N (e.g., between 30° N and 35° N, or between 35° N and 40° N) to a second region between 20° N and 30° N (e.g., between 20° N and 25° N, or between 25° N and 30° N). Herein, the transition is from a region with a longer day to a shorter day. In particular embodiments, a transition to a region with a shorter day may be achieved as a result of an allelic combination that increases the flowering time of an edited plant relative to the control plant.

As an example, based on the allelic combination, an edited non-naturally occurring soybean plant may be transitioned from growth in a first region between 20° N and 30° N (e.g., between 20° N and 25° N, or between 25° N and 30° N) to a second region between 30° N and 40° N (e.g., between 30° N and 35° N, or between 35° N and 40° N), or between 40° N and 50° N (e.g., between 40° N and 45° N, or between 45° N and 50° N). As another example, based on the allelic combination, an edited non-naturally occurring soybean plant may be transitioned from growth in a first region between 30° N and 40° N (e.g., between 30° N and 35° N, or between 35° N and 40° N) to a second region between 40° N and 50° N (e.g., between 40° N and 45° N, or between 45° N and 50° N). Herein, the transition is from a region with a shorter day to a longer day. In particular embodiments, a transition to a region with a longer day may be achieved as a result of an allelic combination that decrease the flowering time of an edited plant relative to the control plant.

One example embodiment of a method of establishing where a soybean plant, or seed thereof, should be grown, comprises: (a) introducing, via genome modification using a site directed nuclease, a mutation at an E1 locus and an E1LB locus of a genome of a soybean plant; (b) selfing the plant for one or more generations to generate a progeny plant that is homozygous at each of the E1 locus and the E1LB locus; (c) obtaining DNA from said progeny plant; (d) determining an allelic combination of said progeny plant via a first assay of the DNA indicative of a type of mutation introduced at the E1 locus and a second assay of the DNA indicative of a type of mutation introduced at the E1LB locus; and (e.) assigning a change in flowering time of the plant based on the determined allelic combination, wherein the change in flowering time is relative to a control plant not comprising the allelic combination. Another example embodiment of the method comprises editing, via a site directed nuclease, at an E1 locus and an E1LB locus of a genome of a soybean plant; isolating DNA from an edited progeny of the soybean plant; determining an allelic combination of said edited progeny plant via a first DNA assay indicative of a type of mutation introduced at the E1 locus and a second DNA assay indicative of a type of mutation introduced at the E1LB locus; and assigning a change in flowering time of the progeny based on the determined allelic combination, wherein the change in flowering time is relative to a control plant not comprising the allelic combination. A further embodiment of the method comprises editing an endogenous E1 gene and E1LB gene of a genome of a soybean plant; selfing the plant for one or more generations to generate a progeny plant that is homozygously edited at each of the E1 gene and the E1LB gene; obtaining DNA from said progeny plant; determining an allelic combination of said progeny plant via a first assay of the DNA indicative of a type of mutation introduced at the E1 gene and a second assay of the DNA indicative of a type of mutation introduced at the E1LB gene; and assigning a change in flowering time of the plant based on the determined allelic combination, wherein the change in flowering time is relative to a control plant not comprising the allelic combination.

Another example embodiment of a method of establishing where a soybean plant, or seed thereof, should be grown, comprises: a. introducing, via genome modification using a site directed nuclease, a mutation at an E1 locus and/or an E1LB locus of a genome of a soybean plant (e.g., into only the E1 locus, only the E1LB locus, or each of the E1 and E1LB loci); b. selfing the plant for one or more generations to generate a progeny plant that is homozygous for the mutation at said E1 locus and/or said E1LB locus; c. obtaining DNA from said progeny plant; d. determining an allelic combination of said progeny plant via an assay indicative of the mutation introduced at said E1 locus and/or said E1LB locus; and e. assigning a change in flowering time and/or maturity time to the progeny plant based on the determined allelic combination, wherein the change in flowering time and/or maturity time is relative to a control plant not comprising the determined allelic combination. For example, the mutation is introduced at a nuclear localization signal (NLS) of the E1 locus and/or the E1LB locus. In particular examples, the mutation in introduced at a basic domain of the NLS of the E1 locus and/or the E1LB locus. In particular examples, the mutation in introduced at a second of two basic domains of a nuclear localization signal (NLS) of the E1 locus and/or the E1LB locus, wherein the second basic domain is downstream relative to a first basic domain of the NLS. In embodiments, the introducing comprises transforming a plant cell with an expression cassette comprising: (i) a nucleic acid that encodes the site-directed nuclease; and (ii) a nucleic acid that encodes at least one guide RNA (gRNA) directed to a target sequence at said E1 locus and/or said E1LB locus. In some examples, the at least one gRNA is directed to a target sequence comprising a second basic domain of the nuclear localization signal (NLS) at one of the E1 locus and the E1LB locus. In other examples, the at least one gRNA is directed to a target sequence comprising a second basic domain of the nuclear localization signal (NLS) at each of the E1 locus and the E1LB locus. In some examples, the nucleic acid that encodes the site-directed nuclease is operably linked to a first promoter of the expression cassette and the nucleic acid that encodes the at least one gRNA is operably linked to a second promoter of the expression cassette, wherein the first promoter and the second promoter are different promoter sequences or have a common promoter sequence. In embodiments, the expression cassette further comprises an enhancer operably linked to the first promoter or the second promoter for enhancing the transcription of sequence operably linked to the promoter. As non-limiting examples, the site directed nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9-cytidine deaminase, chimeric Cas9-adenine deaminase, chimeric FEN1-Fokl, and Mega-TALs, a nickase Cas9 (nCas9), chimeric dCas9 non-Fokl nuclease and dCpf1 non-Fokl nuclease. In some examples, the mutation introduced into the E1 locus and/or the E1LB locus is selected from the group consisting of an allele replacement, one or a plurality of base insertions, one or a plurality of base deletions.

In particular examples, the allelic combination comprises one or more of: a mutant E1 allele resulting in a loss of function of an E1 protein; a mutant E1 allele resulting in partial function of the E1 protein; a mutant E1LB allele resulting in a loss of function of an E1LB protein; and a mutant E1LB allele resulting in partial function of the E1LB protein. For example, the mutant allele at the E1LB locus may correspond to any one of SEQ ID NOS: 15, 17, 19, 21 and 23; or encode a mutant E1LB protein corresponding to any one of SEQ ID NOS: 16, 18, 20, 22 and 24. In other examples, the mutant allele at the E1 locus corresponds to any one of SEQ ID NOS: 5, 7, 9, 11 and 13; or encodes a mutant E1 protein corresponding to any one of SEQ ID NOS: 6, 8, 10, 12 and 14.

In embodiments, the progeny plant has a modified flowering time and/or maturity time relative to the control plant, and wherein the control plant comprises a wild-type allele at each of the E1 locus and E1LB locus. In some examples, assigning a changing in flowering time and/or maturity time comprises assigning a number of days by which the flowering time and/or maturity time is shortened for the progeny plant relative to the control plant. In particular embodiments, assigning a change in the flowering time comprises reducing a number of days between a VE stage and an R1 stage of the progeny plant relative to the control plant, and/or wherein assigning a change in the maturity time comprises reducing a number of days between an R1 stage and an R7 (or R8) stage of the progeny plant relative to the control plant. In embodiments, introducing the mutations further comprises regenerating a transformed TO plant from the transformed plant cell, the transformed TO plant having a plurality of T1 seed, wherein the plurality of T1 seed contain a plurality of unique edits in the E1 locus and/or the E1LB locus; growing a plurality of T1 plants from the T1 seed; and selfing the T1 plants for one or more generations to obtain a progeny plant that is homozygous for the introduced mutation at the E1 locus and/or the E1LB locus. The, wherein determining the allelic combination comprises sequencing the E1 locus and the E1LB locus of the progeny plant; sequencing the E1 locus and the E1LB locus of the TO plant; and aligning the E1 locus and the E1LB locus sequence of the progeny plant with the corresponding sequence of the progeny plant to determine the mutation introduced into the E1 locus and/or the E1LB locus.

Non-limiting embodiments of the invention comprise methods of establishing where a soybean plant, or seed thereof, should be grown. In embodiments, the method comprises: a. introducing, via genome modification using a site directed nuclease, a mutation at an E1 locus and an E1LB locus of a genome of a soybean plant; b. selfing the plant for one or more generations to generate a progeny plant that is homozygous at each of the E1 locus and the E1LB locus; c. obtaining DNA from said progeny plant; d. determining an allelic combination of said progeny plant via a first assay of the DNA indicative of a type of mutation introduced at the E1 locus and a second assay of the DNA indicative of a type of mutation introduced at the E1LB locus; and e. assigning a change in flowering time of the plant based on the determined allelic combination, wherein the change in flowering time is relative to a control plant not comprising the allelic combination. In some embodiment, the editing comprises introducing a mutation at a nuclear localization signal (NLS) at the E1 locus and the E1LB locus. In other embodiments, the editing comprises introducing a mutation at a basic domain of the NLS at the E1 locus and the E1LB locus. In particular embodiments, the editing comprises introducing a mutation at a second of two basic domains of the NLS at the E1 locus and the E1LB locus, wherein the second basic domain is downstream relative to a first basic domain of the NLS. In embodiments, the assigning a change in flowering time comprises assigning a relative maturity group value relative to the control plant not comprising the allelic combination, wherein optionally the control plant comprises a wild-type allele at each of the E1 locus and E1LB locus. In other embodiments, assigning a changing in flowering time comprises assigning a number of days by which the flowering time is advanced or retarded for the soybean plant relative to the control plant.

Non-limiting embodiments of the invention also comprise expression cassettes used for editing the plant cell. Example embodiments comprise transforming a plant cell with an expression cassette comprising (i) a nucleic acid that encodes the site-directed nuclease; and (ii) a nucleic acid that encodes at least one guide RNA (gRNA) directed to a target sequence. In embodiments, the at least gRNA is directed to a target sequence comprising a nuclear localization signal (NLS) at the E1 locus and the E1LB locus. In embodiments, the at least gRNA is directed to a target sequence comprising a second basic domain of the nuclear localization signal (NLS) at the E1 locus and the E1LB locus. In embodiments, the nucleic acid that encodes the site-directed nuclease is operably linked to a first promoter and wherein the nucleic acid that encodes the at least one gRNA is operably linked to a second promoter. In further embodiments of the expression cassette, an enhancer is operably linked to the first promoter or the second promoter. In embodiments, the site directed nuclease is selected from the group consisting of meganucleases (MNs), zinc-finger nucleases (ZFNs), transcription-activator like effector nucleases (TALENs), Cas9 nuclease, Cfp1 nuclease, dCas9-Fokl, dCpf1-Fokl, chimeric Cas9-cytidine deaminase, chimeric Cas9-adenine deaminase, chimeric FEN1-Fokl, and Mega-TALs, a nickase Cas9 (nCas9), chimeric dCas9 non-Fokl nuclease and dCpf1 non-Fokl nuclease.

Non-limiting embodiments of the invention also comprise methods of editing plants at the E1 and/or E1Lb loci to create novel alleles and plants with novel allelic combinations which confer the plant with an altered flowering time profile relative to a control plant not comprising the novel allelic combination. Example embodiments of the method of editing comprise introducing into the E1 locus and the E1LB locus a mutation selected from the group consisting of an allele replacement, one or a plurality of base insertions, one or a plurality of base deletions. In further embodiments, the method comprises regenerating a transformed TO plant from the transformed plant cell, the transformed TO plant having a plurality of T1 seed, wherein the plurality of T1 seed contain a plurality of unique edits in the E1 locus and the E1LB locus; growing a plurality of T1 plants from the T1 seed; and selfing the T1 plants for one or more generations to obtain a progeny plant that is homozygous at the E1 locus and the E1LB locus.

Non-limiting embodiments of the invention also comprise methods of determining the novel allelic combination. In example embodiments, the method comprises sequencing the E1 locus and the E1LB locus of the progeny plant; sequencing the EL locus and the E1LB locus of the TO plant; aligning the E1 locus and the E1LB locus sequence of the progeny plant with the corresponding sequence of the progeny plant to determine the type of mutation introduced into the E1 locus and the E1LB locus. Example embodiments of the allelic combination comprise a loss of function E1 allele or a partial function E1 allele; and a loss of function E1LB allele or a partial function E1LB allele. Still other combinations are possible.

Non-limiting embodiments of the invention further include methods of producing a soybean plant with a modified flowering time. Example embodiments comprise introducing an edit into an E1 gene of a plant cell to generate a mutant E1 allele having reduced function of E1 protein relative to a wild-type E1 allele; introducing another edit in an E1LB gene of the plant cell to produce a mutant E1LB allele having reduced function of E1LB protein relative to a wild-type E1LB allele; regenerating an edited plant from the edited plant cell; and selfing the edited plant to obtain an edited progeny comprising having allelic combination comprising the mutant E1 allele and the mutant E1LB allele, wherein the edited progeny has a flowering time that is modified relative to the flowering time of a control plant comprising the wild-type EL allele and/or the wild-type E1LB allele. In embodiments, introducing the edit into the E1 gene comprises introducing a plurality of base pair deletions into the E1 gene, wherein the mutant E1 allele encodes a frameshifted E1 protein or a truncated E1 protein; and introducing the edit into the E1LB gene comprises introducing a plurality of base pair deletions into the E1LB gene, wherein the mutant E1LB allele encodes a frameshifted E1LB protein or a truncated E1LB protein. In particular embodiments, the truncated E1 protein has no functional activity relative to a wild-type E1 protein, the in-frame deleted mutant E1 protein has partial functional activity relative to the wild-type E1 protein, the truncated E1LB protein has no functional activity relative to a wild-type E1LB protein, and wherein the in-frame deleted mutant E1LB protein has partial functional activity relative to the wild-type E1LB protein. In embodiments, the introducing the edit into the E1 gene comprises introducing the edit in a basic domain of a nuclear localization signal of the E1 gene; and introducing the edit into the E1LB gene comprises introducing the edit in the basic domain of the nuclear localization signal of the E1LB gene. In embodiments, introducing the edit into the E1 gene and the E1LB gene comprises transforming the plant cell with an expression cassette comprising: (i) a nucleic acid that encodes a site-directed nuclease; (ii) a nucleic acid that encodes at least one guide RNA (gRNA) directed to a target sequence; and (iii) at least one promoter operably linked to the site-directed nuclease. In embodiments, the expression cassette further comprises another promoter operably linked to the at least one gRNA and optionally an enhancer element operably linked to the at least one promoter. In further embodiments, the at least one gRNA is directed to a target sequence comprising the nuclear localization signal (NLS) at the E1 locus and the E1LB locus.

Other example embodiments of a method of producing a soybean plant with a modified flowering time comprise introducing an edit into one or more of an E1 gene and an E1LB gene of a soybean plant cell to generate a mutant E1 allele and/or a mutant E1LB allele having reduced function relative to a corresponding wild-type allele (e.g., generate only a mutant E1 allele while leaving the E1LB allele as wild-type, generating only a mutant E1LB allele while leaving the E1 allele as wild-type, or generating each of a mutant E1 and E1LB allele); regenerating an edited plant from the edited plant cell; and selfing the edited plant to obtain an edited progeny plant comprising having a non-naturally occurring allelic combination comprising one or more of the mutant E1 allele and the mutant E1LB allele, wherein the edited progeny plant has a flowering time and/or maturity time that is modified relative to the flowering time and/or maturity time of a control plant comprising the wild-type E1 allele and the wild-type E1LB allele. In particular embodiments, the introducing the edit into the E1 gene comprises introducing a plurality of base pair deletions into the E1 gene, wherein the mutant E1 allele corresponds to any one of SEQ ID NOS: 5, 7, 9, 11 and 13 and encodes an in-frame deletion mutant E1 protein or a truncated E1 protein corresponding to any one of SEQ ID NOS: 6, 8, 10, 12 and 14. In other particular embodiments, introducing the edit into the E1LB gene comprises introducing a plurality of base pair deletions into the E1LB gene, wherein the mutant E1LB allele corresponds to any one of SEQ ID NOS: 15, 17, 19, 21 and 23 and encodes an in-frame deletion mutant E1LB protein or a truncated E1LB protein corresponding to any one of SEQ ID NOS: 16, 18, 20, 22 and 24. In these embodiments, the truncated E1 protein has no functional activity relative to a wild-type E1 protein, the in-frame deletion mutant E1 protein has partial functional activity relative to the wild-type E1 protein, the truncated E1LB protein has no functional activity relative to a wild-type E1LB protein, and the in-frame deletion mutant E1LB protein has partial functional activity relative to the wild-type E1LB protein. In embodiments, the edit in the E1 gene is introduced into a second basic domain of a nuclear localization signal (NLS) of the E1 gene; and the edit into the E1LB gene is introduced in the second basic domain of the nuclear localization signal (NLS) of the E1LB gene. In embodiments, introducing the edit into the E1 gene and/or the E1LB gene comprises transforming the plant cell with an expression cassette comprising (i) a nucleic acid that encodes a site-directed nuclease operably linked to a promoter, the promoter optionally further linked to an enhancer configured to enhance transcription of the site directed nuclease by the promoter; and (ii) a nucleic acid that encodes at least one guide RNA (gRNA) directed to a target sequence in the second basic domain of the NLS of the E1 gene and/or the E1LB gene.

In particular embodiments, the flowering time of the edited progeny plant is shorter than the flowering time of the control plant. In further embodiments, a relative maturity group value of the edited progeny plant is different from the relative maturity group value of the control plant.

Non-limiting embodiments of the invention further include the edited progeny plant created by the methods disclosed above, as well as a further progeny plants of the edited progeny plant, such as those obtained by breeding or selfing. In embodiments, the flowering time of the edited progeny plant is modified (e.g., shorter or longer) than the flowering time of the control plant. In embodiments, a relative maturity group value of the edited progeny plant is different from the relative maturity group value of the control plant.

Non-limiting embodiments of the invention further comprise non-naturally occurring soybean plants having a modified flowering time and comprising a novel allele combination. In embodiments, the novel allele combination is selected from the group comprising: a mutant E1LB allele resulting in partial expression of E1LB protein and a wild-type E1 allele; a mutant E1LB allele resulting in loss of expression of E1LB protein and a wild-type E1 allele; a mutant E1LB allele resulting in partial expression of E1LB protein and a mutant E1 allele resulting in partial expression of E1 protein; a mutant E1LB allele resulting in loss of expression of E1LB protein and a mutant E1 allele resulting in partial expression of E1 protein; a mutant E1LB allele resulting in partial expression of E1LB protein and a mutant E1 allele resulting in loss of expression of E1 protein; and a mutant E1LB allele resulting in loss of expression of E1LB protein and a mutant E1 allele resulting in loss of expression of E1 protein, a wild-type E1LB allele and a mutant E1 allele resulting in loss of expression of E1 protein, and a wild-type E1LB allele and a mutant E1LB allele resulting in partial expression of E1 protein, wherein the flowering time of the non-naturally occurring soybean plant is modified relative to a control plant comprising the wild-type E1 allele and a wild-type E1LB allele. In embodiments, the mutant E1LB allele resulting in partial expression or the mutant E1LB allele resulting in loss of expression are introduced by editing an E1LB gene of the plant using a DNA modification enzyme.

In embodiments, the mutant E1 allele resulting in partial expression or the mutant E1 allele resulting in loss of expression are introduced by editing an E1 gene of the plant using a DNA modification enzyme.

Non-limiting embodiments of the invention comprise modified soybean plants, or plant parts thereof. In embodiments, the modified soybean plant, or plant part thereof, comprises a non-naturally occurring mutant allele at an E1 locus and/or a E1Lb locus, wherein the non-naturally occurring mutant allele is introduced via genome modification using a site directed nuclease. In particular embodiments, said non-naturally occurring mutant allele is a homozygous mutant allele. In example embodiments, the modified soybean plant, or plant part thereof, comprises a non-naturally occurring mutant allele at each of the E1 locus and the E1Lb locus, wherein both of said E1 locus and said E1Lb locus comprise homozygous mutant alleles. In embodiments, the mutant allele exhibits a reduction of expression or enzymatic activity relative to an unmodified, wild-type E1 or E1LB gene allele. In particular embodiments, the mutant allele at the E1 locus or the E1LB locus comprises a mutation in a second basic domain of a nuclear localization signal (NLS) of a protein encoded by the gene. In embodiments, the mutant allele at the E1 locus and/or the E1LB locus comprises one or more mutation types selected from the group consisting of a nonsense mutation, an in-frame deletion mutation, a missense mutation, a frameshift mutation, a splice-site mutation, and any combination thereof.

In non-limiting embodiments of the modified soybean plant, or plant part thereof, the mutant allele at the E1 locus results in one of the following: an E1 protein truncation, a non-functional E1 protein, an E1 protein with reduced function, a premature stop codon in the E1 gene, and an in-frame deletion in the E1 gene; and wherein said mutant allele at the E1LB locus results in one of the following: an E1LB protein truncation, a non-functional E1LB protein, an E1LB protein with reduced function, a premature stop codon in the E1LB gene, and an in-frame deletion in the E1LB gene. In particular embodiments of the modified soybean plant, or plant part thereof, the modified plant has a smaller flowering time that a control plant comprising an unmodified wild-type E1 and/or wild-type E1LB gene allele, such as smaller number of days between a VE stage and an R1 stage of the modified plant relative to the control plant. In additional or alternative embodiments, the modified plant has a smaller maturity time than a control plant comprising an unmodified wild-type E1 and/or wild-type E1LB gene allele, such as a smaller number of days between an R1 stage and an R7 or an R8 stage of the modified plant relative to the control plant.

In particular embodiments of the modified soybean plant, or plant part thereof, the homozygous mutant allele comprises a homozygous mutation at the E1 locus selected from the group consisting of: deletion of 8 bp from position 147 to 154 of the E1 gene; deletion of 13 bp from position 144 to 156 of the E1 gene; deletion of 25 bp from position 131 to 155 of the E1 gene; deletion of 9 bp from position 146 to 154 of the E1 gene; and substitution of 15 bp from position 145 to 159 of the E1 gene, wherein said position is with reference to the E1 gene of SEQ ID NO. 1. In embodiments, the homozygous mutation at the E1 locus corresponds to any one of SEQ ID NOS: 5, 7, 9, 11 and 13; or encodes a mutant E1 protein corresponding to any one of SEQ ID NOS: 6, 8, 10, 12 and 14.

In other embodiments of the modified soybean plant, or plant part thereof, the homozygous mutant allele comprises a homozygous mutation at the E1LB locus selected from the group consisting of: deletion of 7 bp from position 149 to 155 of the E1LB gene; deletion of 3 bp from position 149 to 151 of the E1LB gene; deletion of 139 bp from position 13 to 151 of the E1LB gene; deletion of 6 bp from position 149 to 154 of the E1LB gene; and deletion of 9 bp from position 148 to 156 of the E1LB gene, wherein said position is with reference to the E1LB gene of SEQ ID NO. 3. In embodiments, the homozygous mutation at the E1LB locus corresponds to any one of SEQ ID NOS: 15, 17, 19, 21 and 23; or encodes a mutant E1LB protein corresponding to any one of SEQ ID NOS: 16, 18, 20, 22 and 24.

In other embodiments, a non-naturally occurring soybean plant having a modified flowering time comprises an allele combination selected from the group comprising: (a) a mutant E1LB allele resulting in partial expression of E1LB protein and a wild-type E1 allele; (b) a mutant E1LB allele resulting in loss of expression of E1LB protein and a wild-type E1 allele; (c) a mutant E1LB allele resulting in partial expression of E1LB protein and a mutant E1 allele resulting in partial expression of E1 protein; (d) a mutant E1LB allele resulting in loss of expression of E1LB protein and a mutant E1 allele resulting in partial expression of E1 protein; (e) a mutant E1LB allele resulting in partial expression of E1LB protein and a mutant E1 allele resulting in loss of expression of E1 protein; (f) a mutant E1LB allele resulting in loss of expression of E1LB protein and a mutant E1 allele resulting in loss of expression of E1 protein; (g) a mutant E1 allele resulting in partial expression of E1 protein and a wild-type E1LB allele; and (h) a mutant E1 allele resulting in loss of expression of E1 protein and a wild-type E1LB allele; wherein the flowering time of the non-naturally occurring soybean plant is modified relative to a control plant comprising the wild-type E1 allele and a wild-type E1LB allele.

In particular embodiments, the mutant E1LB allele corresponds to any one of SEQ ID NOS: 15, 17, 19, 21 and 23; or encodes a mutant E1LB protein corresponding to any one of SEQ ID NOS: 16, 18, 20, 22 and 24; and the mutant E1 allele corresponds to any one of SEQ ID NOS: 5, 7, 9, 11 and 13; or encodes a mutant E1 protein corresponding to any one of SEQ ID NOS: 6, 8, 10, 12 and 14. In embodiments, the mutant E1LB allele resulting in partial expression or the mutant E1LB allele resulting in loss of expression are introduced by editing an E1LB gene of the plant using a DNA modification enzyme. In other embodiments, the mutant E1 allele resulting in partial expression or the mutant E1 allele resulting in loss of expression are introduced by editing an E1 gene of the plant using a DNA modification enzyme.

Non-limiting embodiments further comprise a method of breeding comprising: crossing the non-naturally occurring soybean plant disclosed above with a different soybean plant not comprising the allele combination of the non-naturally occurring soybean plant; and selecting a progeny plant having the modified flowering.

### EXAMPLES

The following examples provide illustrative embodiments. In light of the present disclosure and the general level of skill in the art, those of skill will appreciate that the following examples are intended to be exemplary only and that numerous changes, modifications, and alterations can be employed without departing from the scope of the presently disclosed subject matter.

### Example 1: Modification of Endogenous E1 and E1LB Gene Sequences in the Genome of a Plant Cell by Delivering LbCas12a Endonuclease and Guide RNA Expression Cassettes

In one aspect of the invention, there is provided a method of generating allelic variations of E1 and E1LB genes having altered expression levels and patterns relative to endogenous versions of the genes. The allelic variations include allelic variants of E1 and E1LB genes with reduced activity of the E1 or E1LB proteins (herein also referred to as partial function variants) and allelic variants having abolished activity of the E1 or E1LB protein (herein also referred to as loss of function variants), in any plant species containing a E1 or E1LB gene. The method comprises using the DNA sequence of the second basic domain of the nuclear localization signal (NLS) of the proteins encoded by E1 and E1LB genes using methods that are well tested and described for plants. According to these well-established protocols, a guide RNA based on direct sequence homology to the target region of the E1 and/or E1LB gene (second basic domain of NLS, for example) targeted for change (e.g. targeted deletion of a functional region, insertion of a stop codon, insertion of a frame-shift mutation, or any other change in the gene and regulatory region designed to influence the expression of the gene) is synthesized or encoded in a vector for expression in plants, preferably together with a gene encoding LbCas12a protein (although in some embodiments, LbCas12a can also be encoded in a separate vector).

Construction of LbCas12a expression vectors and targeting donors have been described before (H. Puchta et. al., incorporated by reference in its entirety herein). In binary vector 25462 (FIG. 2), the Arabidopsis codon-optimized and catalytically inactive Lachnospiraceae bacterium Cas12a (hereafter dLbCas12a, also k known as dLbCpf1) was driven under the control of Arabidopsis EF-1 alpha A1 promoter with an eFMV enhancer to drive constitutive expression of the Lb12Cas12a (prAtEF1aA1) followed by NOS terminator. A nuclear localization signal was also incorporated into the C-terminus of LbCas12a to improve its targeting to nucleus.

As shown in FIG. 2A, a gRNA expression cassette comprising a soy ubiquitin promoter (prGmUbi) operably linked at the 3′-end to coding sequences for a gRNA and gRNA scaffolds were synthesized by GenScript (www.genscript.com) and cloned into binary vector 25462 which included an aadA gene driven by Soybean EF promoter as the selection marker.

The design of guide RNAs is well known to those skilled in the art. The target was GmE1 (SEQ ID NO: 1). Typically, guide RNA design is specific for each gene sequence and for the desired changes to be made. For example, if only E1 or E1Lb sequences need to be targeted that is predicted to have the desired effect, such as reducing gene function. However, in this case, the same gRNA was used for GmE1Lb target given their ˜93% sequence homology. If all members of a gene family are to be targeted, for example if they have redundant functions such as in the case of E1 and E1Lb, then a conserved sequence specific to those genes can be targeted to make the desired changes. In this case, the target was the conserved second basic domain of the NLS of E1 and E1Lb.

Details of the binary vector 25462 are as follows:
bNLB insertion_seq 12,219 12,348 130 reverse Left border region of T-DNA of Agrobacterium tumefaciens nopaline ti-plasmid
bNRB insertion_seq 4 143 140 reverse Right border region of T-DNA of Agrobacterium tumefaciens nopaline ti-plasmid
cLbCas12a CDS 1,871 5,743 3,873 forward This LbCas12a is an Arabidopsis codon-optimized version.
cRepA CDS 14,471 15,544 1,074 forward cRepA-01 with A to G at nt735
cSpec CDS 12,628 13,416 789 forward Also called aadA; gene encoding the enzyme aminoglycoside 3′adenyltransferase that confers resistance to spectinomycin and streptomycin for maintenance of the vector in E. coli and Agrobacterium.
cVirG CDS 13,716 14,441 726 forward virG (putative) from pAD1289 with TTG start codon. Described in Hansen et al. 1994, PNAS 91:7603-7607
eFMV enhancer 157 698 542 forward Figwort mosaic virus (FMV) enhancer GmE1 target misc_feature 8,072 8,094 23 forward
iAtEF1aAluintron 1,252 1,864 613 forward UTR intron of AtEF1aA1.
iGmEF intron 9,570 10,476 907 forward The first intron of the soybean elongation factor (EF) gene
iGmUbi1 intron 7,470 8,001 532 forward The 5′ UTR intron of GmaSOYUBI gene.
oCOLE rep_origin 16,669 17,475 807 reverse The ColE1 origin of replication functional in E. coli derived from pUC19
oVS1 rep_origin 15,587 15,991 405 forward origin of replication in Agrobacterium tumefaciens host
Complimentary strand sequence of 1st 6 bp in most Cpf1 CrRNAs (LbCpf1, AsCpF1, and FnCpf1), to allow for hammerhead self-cleavage at the correct position just prior to the subsequent CrRNA
prAtEF1aA1 promoter 705 1,864 1,160 forward Promoter from Arabidopsis thaliana EF-1 alpha A1 gene for elongation factor
prGmEF promoter 8,424 10,487 2,064 forward
Translation elongation factor EF-1 alpha/Tu promoter, including the first intron and neighboring UTR from soybean (williams 82)
prGmUbi1 promoter 6,004 8,001 1,998 forward Ubiquitin 1 promoter candidate sourced from Soy Williams 82
prVirG promoter 13,511 13,641 131 forward virG promoter (Winans J. Bact. 172:2433-38 (1990)) composed of two promoter elements, one responsive to acetosyringone and phosphate-starvation (bp 45 to 83) and another to medium acidification (86 to 128)
rHDV misc_RNA 8,095 8,162 68 forward A sequence encoding a self-cleavable ribozyme from hepatitis delta virus (HDV).
rHH misc_RNA 8,014 8,050 37 forward Hammerhead RNA; a conserved sequence motif from Satellite RNAs of certain viruses, including Tobacco ringspot virus, responsible for self-cleavage. Pairing sequence needs to be added at 5′ end to ensure proper cleavage.
rHHLbCrRNA misc_RNA 8,008 8,050 43 forward Hammerhead RNA; a conserved sequence motif from Satellite RNAs of certain viruses, including Tobacco ringspot virus, responsible for self-cleavage. Pairing sequence from Cas12a CrRNA was added at 5′ end to ensure proper cleavage
rLbCrRNA-01 misc_RNA 8,051 8,071 21 forward The scaffold crRNA of LbCpf1 (currently known as Cas12a), also called direct repeat (DR) of guide RNA.
rLbgRNACas12aGmEl-01 misc_RNA 8,051 8,094 44 forward
Cas12a (previously known as Cpf1) guide RNA including direct repeat of LbCrRNA targeting ggacatcaaggagaagattctgc of GmE1
Start misc_feature9,507 9,507 1 none transcription start site startmisc_feature9,507 9,507 1 none transcription start site</nobr></html>TATA-box TATA_signal 1,166 1,176 11 forward <html><nobr>TATA box of AtEF1aA1 promoter
tNOS terminator 8,163 8,415 253 forward synthetic Nopaline synthetase terminator
tNOS terminator 5,751 6,003 253 forward synthetic Nopaline synthetase terminator
tPsE9 terminator 11,551 12,193 643 forward 3′-UTR of the pea (Pisum sativum) rib-1,5-bisphospate carboxylase (rbcS2) small subunit E9
TSSmisc_feature7,346 7,346 1 none Transcription starting site u5GmEF-01 5′UTR 9,507 9,569 63 forward 5′ UTR of the soybean elongation factor (EF) gene, before intron
u5GmEF-02 5′UTR 10,477 10,487 11 forward 5′ UTR of the soybean elongation factor (EF) gene
uAtL-EF1aA1 5′UTR 1,197 1,251 55 forward 5′-UTR of AtEF1aA1 (Arabidopsis elongation factor-1 alpha), leader sequence of AtEF1aA1 gene uAtRNAligase-01 3′UTR 705 959 255 forward 3′ UTR of Arabidopsis thaliana RNL (RNA LIGASE)
xAtCTP2-01 transit_peptide 10,492 10,719 228 forward Chloroplast transit peptide of Arabidopsis thaliana 3-phosphoshikimate 1-carboxyvinyltransferase/5-enolpyruvylshikimate-3-phosphate/EPSP synthase (AT2G45300)
xLinker-08 misc_feature 1,895 1,984 90 forward The poly peptide linker is based on GGGGS unit which is repeated 6 units to generate 30 aa protein linker, it was soybean codon optimized
xSGGSlinker-05 misc_feature 5,696 5,707 12 forward 4 aa linker of fusion protein from paper: Nature Biotechnology volume 35, pages 438-440 (2017), it was soybean codon optimized
xSGGSlinker-06 misc_feature 5,708 5,719 12 forward 4 aa linker of fusion protein from paper: Nature Biotechnology volume 35, pages 438-440 (2017), it was soybean codon optimized
xSV40NLS-13 misc_feature5,675 5,695 21 forward Nuclear location signal of 7 aa from SV40. It was soybean codon optimized
xSV40NLS-13 misc_feature 1,874 1,894 21 forward Nuclear location signal of 7 aa from SV40. It was soybean codon optimized
xSV40NLS-14 misc_feature 5,720 5,740 21 forward Nuclear location signal of 7 aa from SV40 It was soybean codon optimized.
   (a) The gRNA Design

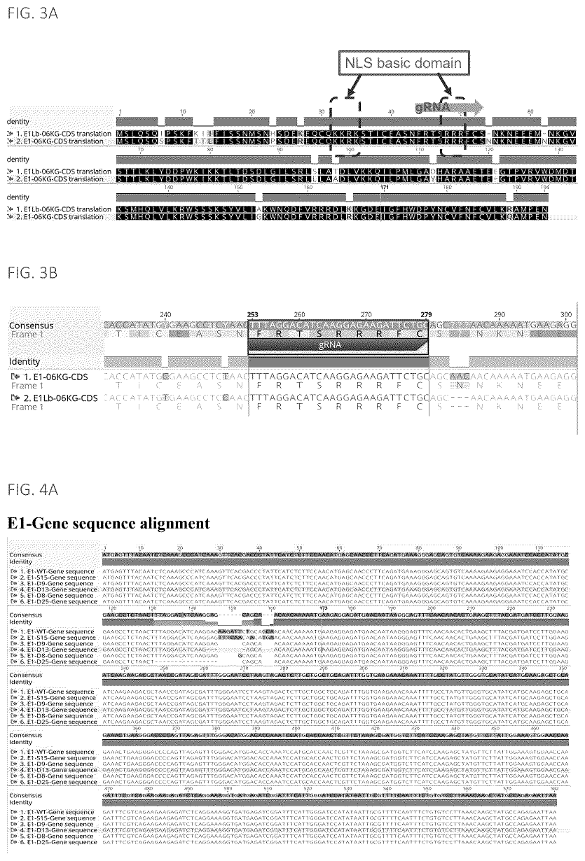

One gRNA was designed to target second basic domain of NLS at both two genes, E1 and E1Lb, as shown in FIG. 3A The target sequence for the gRNA was based on a consensus sequence determined following an alignment of the conserved domains (FIG. 3B). The gRNA sequence was (SEQ ID NO: 37): TTTAGGACATCAAGGAGAAGATTCTGC (PAM: TTTA)

### (b) Novel Alleles of E1 and E1Lb

Novel alleles of E1 and E1Lb created using gene editing included in-frame deletion mutations in the second basic NLS domain of the target gene. Such in-frame deletions are in integral multiples of 3 bp, and include, for example, 6 bp, 9 bp, 12 bp and 15 bp deletions, and result in weak alleles having a reduced expression level relative to their wild-type counterpart. The mutated protein encoded by the in-frame deletion mutation is shorter than the wild-type protein and has reduced activity. Example in-frame deletion alleles and their placement relative to the second basic NLS domain of the target gene are shown at FIG. 6 (alignments shown at the nucleotide and amino acid level). In some embodiments, the in-frame deletions resulted in alleles that were leaky and had reduced E1 or E1Lb activity relative to the wild-type counterpart. In other embodiments, the in-frame deletions resulted in alleles that had no activity.

Novel alleles of E1 and E1Lb created using gene editing also included loss of function (lof) deletion mutations in the second basic NLS domain of the target gene. Such lof deletions include, for example, 4 bp, 7 bp and 8 bp deletions (that is, not integral multiples of 3 bp). Example lof deletion alleles and their placement relative to the second basic NLS domain of the target gene is shown at FIG. 7 (alignments shown at the nucleotide and amino acid level). In some embodiments, the alignments of FIGS. 6 and 7 can be compared to the alignment of FIG. 3A to determine the position of the mutations.

### Example 2: Soybean Transformation Method

Soybean (Glycine max, variety Jack) seeds were sterilized by chlorine gas and imbibed in germination media at 25° C. in the dark. Imbibed seeds were used to prepare explants by trimming off the hypocotyl, removing one cotyledon and leaf primordia, as described in Khan et al. (Method of transforming soybean, WIPO International Publication Number WO2004000006, Dec. 31, 2003; incorporated by reference) and Watts J. et al. (U.S. Pat. No. 9,758,792, incorporated by reference). The shoot apical region and the cot-node region were further wounded with the sharp end of a scalpel blade gently. The prepared explants were immediately infected with Agrobacterium suspension that comprised Agrobacterium tumefaciens strain of EHA101 containing binary vector 25462. With at least 2 hours inoculation, the explants were placed on a co-cultivation medium for 5 days at 23° C. in the dark. After co-cultivation, the explants were preferably transferred to recovery medium without selection agent for about 7 days at 24° C. under 16 hours light/8 hours dark regimen. The recovered explants with the cotyledon were transferred to regeneration media along with glyphosate selection for about 3 weeks. The explants with developing multiple shoots clusters were transferred to elongation medium along with glyphosate selection for shoot elongation. Subcultures to fresh elongation media were performed every 3 weeks until elongated shoots (>3 cm) were long enough to be sampled for molecular analysis.

### Example 3: Molecular Analysis

Before the E0 plants from transformation were sent to greenhouse, DNA extracted from leaves of E0 plants and 06KG wildtype (used as control) were used as template to identify T-DNA insertion copy number, by Taqman assay, and target genes editing by Singer sequencing. The edited events with single copy T-DNA insertion were sent to greenhouse for E1 seeds harvest. 06KG background has a genotype E1E2E3E4 and a relative maturity group of 5.5.

### (a) Target Gene (E1 and E1Lb) Specific Amplification

Two pairs of primers specific to E1 and E1Lb were designed for gene cloning. The PCR amplified DNA fragment length were 1668 bp (E1) and 1647 bp (E1Lb), respectively. PCR specific primers used are listed below in Table 1. PCR reaction and cycle details are provided in Tables 2 and 3, respectively.

TABLE 1
PCR specific primers for E1 and E1Lb

| | | |
|---|---|---|
| Forward primer | E1Lb | GTTATCATGCTTGAAAATGATGATG |
| (SEQ ID NO: 25) | | |
| Reverse primer | E1Lb | GTGCATAGATCTCATGCTAGACCT |
| (SEQ ID NO: 26) | | |
| Forward primer | E1 | TCCATTGTTTGTAGCGCATGGTC |
| (SEQ ID NO: 27) | | |
| Reverse primer | E1 | CATAGATCTCCTGCTAGCTAGACC |
| (SEQ ID NO: 28) | | |

TABLE 2
PCR reaction mixture for E1 and E1Lb gene amplification
PCR reaction mixture for E1 and E1Lb gene amplification

| | | |
|---|---|---|
| Reaction mix | 25 | ul |
| Template | 5 | ul |
| dNTP | 2.5 | ul |
| Buffer | 2.5 | ul |
| DMSO | 0.75 | ul |
| Mg²⁺ | 1.5 | ul |
| Primer (FW/RE) | 0.75 | ul |
| Plus-Neo | 0.5 | ul |
| ddH₂O | 10.75 | ul |

TABLE 3
PCR cycle conditions for E1 and E1Lb
PCR cycle conditions for E1 and E1Lb

| | | | |
|---|---|---|---|
| 95° C. | 30 | s | |
| 95° C. | 15 | s | 35 cycles |
| 58° C. | 20 | s | |
| 68° C. | 1.5 | min | |
| 68° C. | 5 | min | |

### (b) Target Genes Sequencing:

To determine the types of mutation at target sites, E1 and E1Lb specific amplification fragments were used to analyze the target site sequence. PCR products were detected by 1% agarose gel electrophoresis and then sequenced. The E1 and E1Lb gene sequencing primer are shown in Table 4.

TABLE 4
Sequencing primers for E1 and E1Lb

| | | |
|---|---|---|
| Sequencing Primer | E1 | GCTAGTAGTGTAAACACTCAA |
| (SEQ ID NO: 30) | | AACAC |
| Sequencing Primer | E1Lb | TGCCTTTCTACTGGCTATGAC |
| (SEQ ID NO: 29) | | |

### (c) TAQMAN Assay

The T-DNA insertion copy number was identified by Taqman assay. The target gene (Cas12a) and internal housekeeping gene (Adh) amplified specific primers are shown at Table 5, while probe sequences are shown at table 6.

TABLE 5
PCR specific primers for Cas12a
and Adh for use in Taqman assay

| | Primers | Sequence 5′-3′ |
|---|---|---|
| Forward primer | Cas12a | GGGCAAGCTCTACATGTTC |
| (SEQ ID NO: 31) | | CAG |
| Reverse primer | Cas12a | CCGTGGTTGTTCTCGTCGA |
| (SEQ ID NO: 32) | | |
| Forward primer | Adh | AGGTGTGGATCGGGCTGTT |
| (SEQ ID NO: 33) | | |
| Reverse primer | Adh | CATCGTGGACGCATTCGA |
| (SEQ ID NO: 34) | | |

TABLE 6
Probes for Taqman assay

| Probes | Sequence 5′-3′ |
|---|---|
| For Cas12a | CGTGGCTCTTGTCGGAGAAGTCCTTGTT |
| (SEQ ID NO: 35) | |
| For Adh | ACTGGCAGCATCCAAGCCATGGTCT |
| (SEQ ID NO: 36) | |

### Example 4: Events Selection for Phenotyping

Mutations were identified by sequence peaks. Heterozygous mutations showed chaotic peaks after the target site, while wild types and homozygous mutations showed single peaks at the target. The sequences of homozygous mutations were aligned with wild types to further determine the variation of target (FIGS. 4A-B and 5A-B).

### (a) E1 Generation

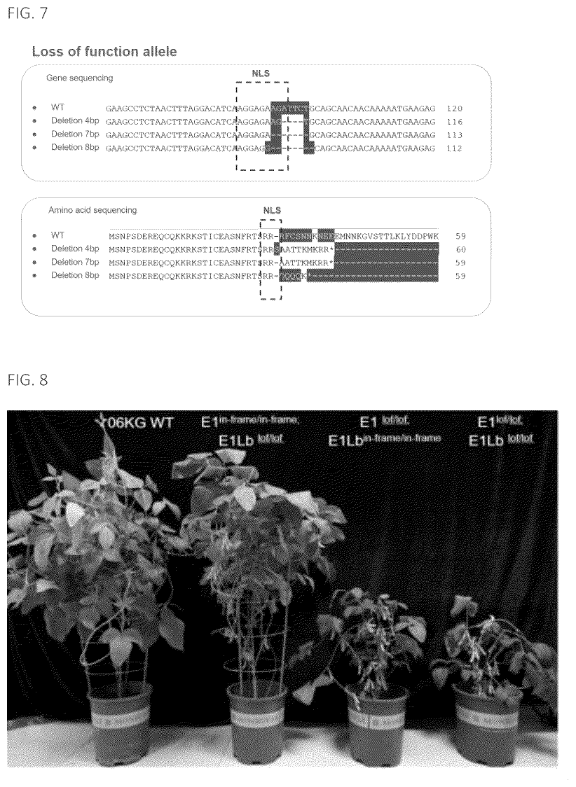

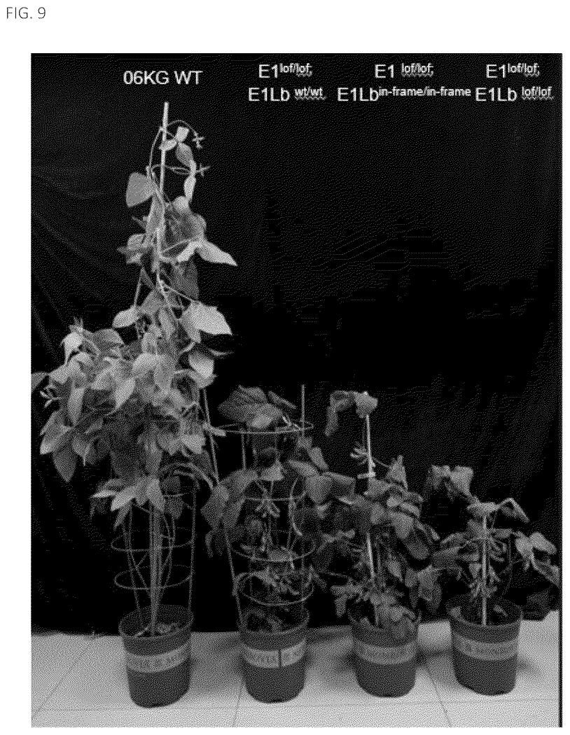

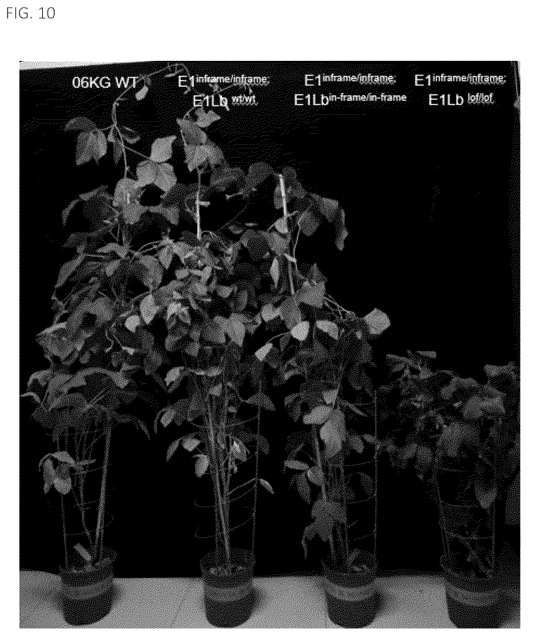

After propagation and segregation, at E1 generation, homozygous mutations of 5 different genotypes, representing 5 different E1 and E1Lb combinations, had been obtained. These edited events had various shortened flowering time when grown in 16 h daylength conditions in a greenhouse with a sow date of July (summer sowing). Phenotype of E1 generation soybean plants comprising novel allelic combinations is shown at FIG. 8. Change in flowering time in the E1 generation plants as a result of the novel allelic combinations is shown at Table 7 below.

TABLE 7
Details of E1 generation homozygous mutants (Abbreviations
used: Lof-Loss of function, in-frame- in-frame deletion
in NLS and partial of function)

| | Flowering | Post Flowering |
|---|---|---|
| | time | time |
| Genotype | (VC-R1) | (R1-R7) |
| E1^{lof/lof;} E1Lb^{lof/lof} | ~27 days | ~55 days |
| E1^{lof/lof;} E1Lb^{in-frame/in-frame} | ~30 days | ~57 days |
| E1^{in-frame/in-frame;} E1Lb^{lof/lof} | ~47 days | ~79 days |
| E1^{in-frame/in-frame;} E1Lb^{in-frame/in-frame} | ~50 days | ~82 days |
| E1^{wt/wt;} E1Lb^{lof/lof} | ~77 days | ~87 days |
| 06KG | ~80 days | ~91 days |

### (b) E2 Generation

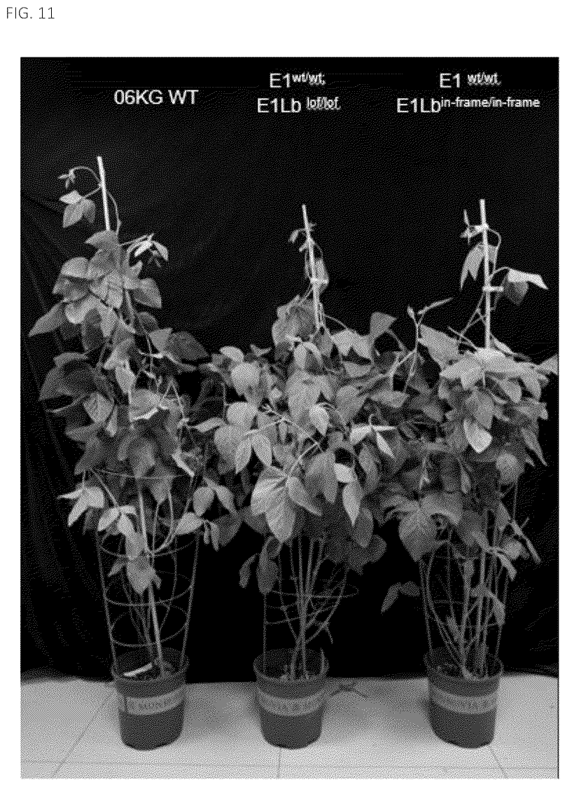

After two generation propagation and segregation, at E2 generation, T-DNA free homozygous mutations of 9 different genotypes, representing 8 different E1 and E1Lb allelic combinations had been obtained and compared to a wild-type allelic combination genotype. Phenotype of E2 generation soybean plants comprising novel allelic combinations is shown at FIGS. 9-11. Details of some example mutants are shown in Table 8.

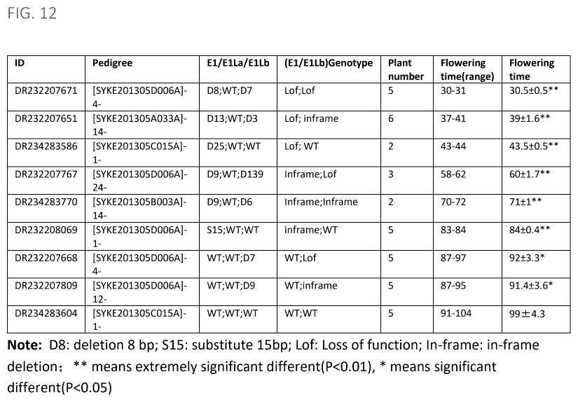

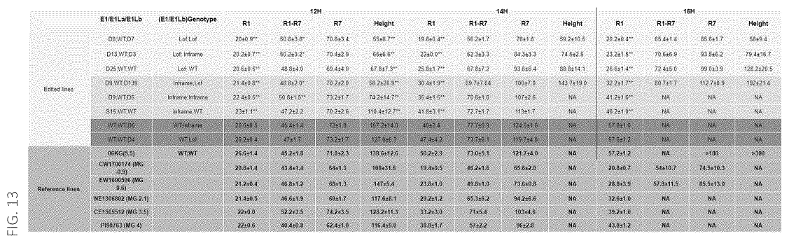

We were able to successfully obtain edited 06KG events with various shortened flowering time. The edits were homozygous mutations and Cas12a free edits. These edited events had various shortened flowering time when grown in 16 h daylength conditions in a greenhouse with a sow date of February (winter sowing) Change in flowering time in the E2 generation plants as a result of the novel allelic combinations is shown at FIG. 12.

TABLE 8
Details of T-DNA free homozygous mutants (Abbreviations
used: Lof-Loss of function)

| | | (E1/E1Lb) | |
|---|---|---|---|
| Mutant ID | E1/E1Lb | Genotype | Description |
| DR232207671 | D8; D7 | E1-Lof; E1Lb-Lof | E1: Deletion 8 bp from CDS 147 to |
| | | | 154 (truncated protein with 54aa) |
| | | | E1Lb: Deletion 7 bp from CDS 149 |
| | | | to 155 |
| DR232207651 | D13; D3 | E1-Lof; E1Lb- | E1: Deletion 13 bp from CDS 144 |
| | | inframe | to 156 |
| | | | (Truncated protein with 56aa) |
| | | | E1Lb: Deletion 3 bp from CDS 149 |
| | | | to 151 |
| DR234283586 | D25; WT | E1-Lof; E1Lb-WT | E1: Deletion 25 bp from CDS 131 |
| | | | to 155 |
| | | | (truncated protein with 54aa) |
| DR232207767 | D9; D139 | E1-Inframe; E1Lb- | E1: Deletion 9 bp from CDS 146 to |
| | | Lof | 154 |
| | | | E1Lb: Deletion 139 bp from CDS |
| | | | 13 to 151 |
| DR234283770 | D9; D6 | E1-Inframe; E1Lb- | E1: Deletion 9 bp from CDS 146 to |
| | | Inframe | 154 |
| | | | E1Lb: Deletion 6 bp from CDS 149 |
| | | | to 154 |
| DR232208069 | S15; WT | E1-inframe; E1Lb- | E1: substitute 15 bp from CDS 145 |
| | | WT | to 159 |
| | | | (AGTTTCAACAACATG replace |
| | | | AGAAGATTCTGCAGC) |
| DR232207668 | WT; D7 | E1-WT; E1Lb-Lof | E1Lb: Deletion 7 bp from CDS 149 |
| | | | to 155 |
| DR232207809 | WT; D9 | E1-WT; E1Lb- | E1Lb: Deletion 9 bp from CDS 148 |
| | | inframe | to 156 |

### Example 5: Phenotyping in Greenhouse

### (a) Plant Materials, Growth Conditions, and Phenotyping

Soybean wildtype 06KG(MG5.5) and edited 06KG events were grown in greenhouse (Beijing) with a sowing date of February (that is, winter). The environment setting in the greenhouse was a day temperature of 28° C. and night temperature of 18° C. in winter. A Sodium lamp was used to supply light from 5 am to 9 μm. The average photon flux was 300 umol/m²/s. The plants were grown under long-day (LD) conditions (16 h light/8 h dark cycle). Flowering time was recorded as the number of days after emergence (DAE) when the first flower opened (R1 stage).

### (b) Phenotyping Data of Different E1L and E1Lb Allelic Combinations in Greenhouse Condition

Phenotyping results of different non-naturally occurring soybean plants have example allelic combinations is shown in Table 9 as well as FIG. 12.

TABLE 9
Phenotyping data of different El and E1Lb allelic combinations
in greenhouse conditions (Abbreviations used: Lof-Loss of
function; in-frame deletion in NLS; FT-Flowering Time)

| | | (E1/E1Lb) | Plant | FT in days | FT in days | Maturity |
|---|---|---|---|---|---|---|
| Mutant ID | E1/E1Lb | Genotype | No. | (range) | (Avg.) | group |
| DR232207671 | D8; D7 | E1-Lof; | 5 | 30-31 | 30.4 ± 0.5 ** | 000 |
| | | E1Lb-Lof | | | | |
| DR232207651 | D13; D3 | E1-Lof; | 6 | 37-41 | 39 ± 1.7 ** | 00 |
| | | E1Lb-inframe | | | | |
| DR234283586 | D25; WT | E1-Lof; | 2 | 43-44 | 43.5 ± 0.5 ** | 0/1 |
| | | E1Lb-WT | | | | |
| DR232207767 | D9; D139 | E1-Inframe; | 3 | 58-62 | 59.7 ± 1.7 ** | 2 |
| | | E1Lb-Lof | | | | |
| DR234283770 | D9; D6 | E1-Inframe; | 2 | 70-72 | 71.0 ± 1.0 ** | 3 |
| | | E1Lb-Inframe | | | | |
| DR232208069 | S15; WT | E1-inframe; | 5 | 83-84 | 83.8 ± 0.4 ** | 4 |
| | | E1Lb-WT | | | | |
| DR232207668 | WT; D7 | E1-WT; | 5 | 87-97 | 92.0 ± 3.4 * | 5 |
| | | E1Lb-Lof | | | | |
| DR232207809 | WT; D9 | E1-WT; | 5 | 87-95 | 91.4 ± 3.6 * | 5 |
| | | E1Lb-inframe | | | | |
| DR234283604 | WT; WT | E1-WT; | 5 | 91-104 | 99.0 ± 3.3 | 5.5 |
| | | E1Lb-WT | | | | |

### Example 6: Non-Naturally Occurring Soybean Plants Comprising Novel Allelic Combinations that Confer the Plant with an Altered Flowering Time and/or Maturity Profile

Based on the phenotyping results (FIGS. 8-11) and the flowering time data (FIG. 12, Table 7 and Table 9), various conclusions were drawn as to the role of the E1 and E1Lb genes, and their novel alleles in flowering development in soybean.

Plants comprising a single E1 loss of function mutation resulted in a significantly earlier flowering time. In embodiments, the flowering time was 55 days earlier than their wild-type counterpart. Plants comprising a single E1 loss function mutation also had a change in plant architecture that make the plant dwarf, likely due to early stem growth termination.

In an E1 loss function genotype background, E1Lb mutations appear to have an additive effect on early flowering time and stem growth termination with the E1Lb lof allele having a larger effect than the E1Lb in-frame allele (compare, for example, in FIGS. 8-12, plants with wild-type (WT) allele background with those having an E1/E1Lb genotype of lof:lof, lof:inframe, and lof:WT).

Plants comprising a single E1 in-frame deletion mutation appeared to promote earlier flowering time, although not as significantly advanced as in E1 lof mutations. In embodiments, the flowering time for single E1 in-frame deletion mutations was 15 days earlier than their wild-type counterpart. Plants comprising a single E1 in-frame deletion mutation appeared to have no change in plant architecture.

In an E1 in-frame deletion mutation genotype background, E1Lb mutations appear to have an additive effect on early flowering time and stem growth termination with the E1Lb lof allele having a larger effect than the E1Lb in-frame allele (compare, for example, in FIGS. 8-12, plants with wild-type (WT) allele background with those having an E1/E1Lb genotype of in-frame:in-frame, in-frame:lof, and in-frame:WT).

Plants comprising a single E1Lb loss function mutation appeared to slightly promote early flowering (in embodiments, 7-8 days earlier) and had no observable impact on plant architecture. Plants comprising a single E1Lb in-frame deletion mutation appeared to slightly promote early flowering (in embodiments, 7-8 days earlier) and had no observable impact on plant architecture. In an E1 WT genotype background, both the E1Lb loss function allele and the E1Lb in-frame deletion allele have similar effect on flowering (compare, for example, in FIGS. 8-12, plants with wild-type (WT) allele background with those having an E1/E1Lb genotype of WT:in-frame, and WT:lof).

Sequence listing

| SEQ ID NO: | AA/NTS | Details |
|---|---|---|
| 1 | NTS | E1, wild-type |
| 2 | AA | E1, wild-type |
| 3 | NTS | E1Lb, wild-type |
| 4 | AA | E1Lb, wild-type |
| 5 | NTS | E1-D8, lof (8 bp deletion, results in loss of function) |
| 6 | AA | E1-D8, lof (premature termination, truncated protein with 54AA) |
| 7 | NTS | E1-D13, lof (13 bp deletion, results in loss of function) |
| 8 | AA | E1-D13, lof (premature termination, truncated protein with 56AA) |
| 9 | NTS | E1-D25, lof (25 bp deletion, results in loss of function) |
| 10 | AA | E1-D25, lof (premature termination, truncated protein with 52AA) |
| 11 | NTS | E1-D9, in-frame (9 bp deletion, results in in-frame deletion) |
| 12 | AA | E1-D9, in-frame (in-frame mutated protein with partial function, |
| | | 190A) |
| 13 | NTS | E1-S15, in-frame (15 bp substitution, results in in-frame deletion) |
| 14 | AA | E1-S15, in-frame (in-frame mutated protein with partial function, |
| | | 193AA) |
| 15 | NTS | E1Lb-D7, lof (7 bp deletion, results in loss of function) |
| 16 | AA | E1Lb-D7, lof (premature termination, truncated protein with |
| | | 57AA) |
| 17 | NTS | E1Lb-D139, lof (139 bp deletion, results in loss of function) |
| 18 | AA | E1Lb-D139, lof (premature termination, truncated protein with |
| | | 13AA) |
| 19 | NTS | E1Lb-D3, in-frame (3 bp deletion, results in in-frame deletion) |
| 20 | AA | E1Lb-D3, in-frame (in-frame mutated protein with partial function, |
| | | 191AA) |
| 21 | NTS | E1Lb-D6, in-frame (6 bp deletion, results in in-frame deletion) |
| 22 | AA | E1Lb-D6, in-frame (in-frame mutated protein with partial function, |
| | | 190AA) |
| 23 | NTS | E1Lb-D9, in-frame (9 bp deletion, results in in-frame deletion) |
| 24 | AA | E1Lb-D9, in-frame (in-frame mutated protein with partial function, |
| | | 189AA) |
| 25 | NTS | E1Lb PCR forward primer |
| 26 | NTS | E1Lb PCR reverse primer |
| 27 | NTS | E1 PCR forward primer |
| 28 | NTS | E1 PCR reverse primer |
| 29 | NTS | E1Lb sequencing primer |
| 30 | NTS | E1 sequencing primer |
| 31 | NTS | Cas12a forward primer |
| 32 | NTS | Cas12a reverse primer |
| 33 | NTS | Adh forward primer |
| 34 | NTS | Adh reverse primer |
| 35 | NTS | Cas12a probe |
| 36 | NTS | Adh probe |
| 37 | NTS | gRNA sequence |

SEQ ID NO: 1 (E1 CDS sequence; WT):
ATGAGTTTACAATCTCAAAGCCCATCAAAGTTCACGACCCTATTCA
TCTCTTCCAACATGAGCAACCCTTCAGATGAAAGGGAGCAGTGTCA
AAAGAAGAGGAAATCCACCATATGCGAAGCCTCTAACTTTAGGACA
TCAAGGAGAAGATTCTGCAGCAACAACAAAAATGAAGAGGAGATGA
ACAATAAGGGAGTTTCAACAACACTGAAGCTTTACGATGATCCTTG
GAAGATCAAGAAGACGCTAACCGATAGCGATTTGGGAATCCTAAGT
AGACTCTTGCTGGCTGCAGATTTGGTGAAGAAACAAATTTTGCCTA
TGTTGGGTGCATATCATGCAAGAGCTGCAGAAACTGAAGGGACCCC
AGTTAGAGTTTGGGACATGGACACCAAATCCATGCACCAACTCGTT
CTAAAGCGATGGTCTTCATCCAAGAGCTATGTTCTTATTGGAAAGT
GGAACCAAGATTTCGTCAGAAGAAGAGATCTCAGGAAAGGTGATGA
GATCGGATTTCATTGGGATCCATATAATTGCGTTTTCAATTTCTGT
GTCCTTAAACAAGCTATGCCAGAGAATTAA
SEQ ID NO: 2 (E1 amino acid sequence; WT):
MSLQSQSPSKFTTLFISSNMSNPSDEREQCQKKRKSTICEASNFRT
SRRRFCSNNKNEEEMNNKGVSTTLKLYDDPWKIKKTLTDSDLGILS
RLLLAADLVKKQILPMLGAYHARAAETEGTPVRVWDMDTKSMHQLV
LKRWSSSKSYVLIGKWNQDFVRRRDLRKGDEIGFHWDPYNCVFNFC
VLKQAMPEN
SEQ ID NO: 3 (E1Lb CDS sequence; WT):
ATGAGTTTACAATCACAAATCCCATCAAAGTTTAAAATAATATTCA
TCTCTTCCAACATGAGCAATCATTCAGATGAGAAGGAGCAGTGTCA
AAAGAAGAGGAAATCCACCATATGTGAAGCCTCCAACTTTAGGACA
TCAAGGAGAAGATTCTGCAGCAACAAAAATGAAGAGGAGATGAACA
AGGGAGTTTCAACAACACTGAAGCTTTATGATGACCCTTGGAAGAT
CAAGAAGACGCTAACCGATAGCGATTTGGGAATCCTAAGTAGACTC
TCGCTGGCTACAGATTTGGTGAAGAAGCAAATTTTGCCTATGTTGG
GTGCAGATCATGCAAGAGCTGCAGAAACTGAAGAAGGGACCCCAGT
TAGAGTTTGGGACATGGACACCAAATCCATGCACCAGCTCGTTCTA
AAGCGATGGTCTTCTTCCAAGAGCTATGTTCTTATTGCAAAGTGGA
ACCAAGATTTCGTGAGAAGAAGAGACCTCAAGAAAGGGGATGAGAT
CGGATTTCATTGGGATCCATATAATTGCGTTTTCAATTTCTGTGTC
CTTAAACGAGCTATGCCAGAGAATTAA
SEQ ID NO: 4 (E1Lb amino acid sequence; WT):
MSLQSQIPSKFKIIFISSNMSNHSDEKEQCQKKRKSTICEASNFRT
SRRRFCSNKNEEEMNKGVSTTLKLYDDPWKIKKTLTDSDLGILSRL
SLATDLVKKQILPMLGADHARAAETEEGTPVRVWDMDTKSMHQLVL
KRWSSSKSYVLIAKWNQDFVRRRDLKKGDEIGFHWDPYNCVFNFCV
LKRAMPEN
SEQ ID NO: 5 (E1-D8 CDS sequence; Lof):
ATGAGTTTACAATCTCAAAGCCCATCAAAGTTCACGACCCTATTCA
TCTCTTCCAACATGAGCAACCCTTCAGATGAAAGGGAGCAGTGTCA
AAAGAAGAGGAAATCCACCATATGCGAAGCCTCTAACTTTAGGACA
TCAAGGAGGCAGCAACAACAAAAATGAAGAGGAGATGAACAATAAG
GGAGTTTCAACAACACTGAAGCTTTACGATGATCCTTGGAAGATCA
AGAAGACGCTAACCGATAGCGATTTGGGAATCCTAAGTAGACTCTT
GCTGGCTGCAGATTTGGTGAAGAAACAAATTTTGCCTATGTTGGGT
GCATATCATGCAAGAGCTGCAGAAACTGAAGGGACCCCAGTTAGAG
TTTGGGACATGGACACCAAATCCATGCACCAACTCGTTCTAAAGCG
ATGGTCTTCATCCAAGAGCTATGTTCTTATTGGAAAGTGGAACCAA
GATTTCGTCAGAAGAAGAGATCTCAGGAAAGGTGATGAGATCGGAT
TTCATTGGGATCCATATAATTGCGTTTTCAATTTCTGTGTCCTTAA
ACAAGCTATGCCAGAGAATTAA
SEQ ID NO: 6 (E1-D8 amino acid sequence; Lof):
MSLQSQSPSKFTTLFISSNMSNPSDEREQCQKKRKSTICEASNFRT
SRRQQQQK
SEQ ID NO: 7 (E1-D13 CDS sequence; Lof):
ATGAGTTTACAATCTCAAAGCCCATCAAAGTTCACGACCCTATTCA
TCTCTTCCAACATGAGCAACCCTTCAGATGAAAGGGAGCAGTGTCA
AAAGAAGAGGAAATCCACCATATGCGAAGCCTCTAACTTTAGGACA
TCAAGAGCAACAACAAAAATGAAGAGGAGATGAACAATAAGGGAGT
TTCAACAACACTGAAGCTTTACGATGATCCTTGGAAGATCAAGAAG
ACGCTAACCGATAGCGATTTGGGAATCCTAAGTAGACTCTTGCTGG
CTGCAGATTTGGTGAAGAAACAAATTTTGCCTATGTTGGGTGCATA
TCATGCAAGAGCTGCAGAAACTGAAGGGACCCCAGTTAGAGTTTGG
GACATGGACACCAAATCCATGCACCAACTCGTTCTAAAGCGATGGT
CTTCATCCAAGAGCTATGTTCTTATTGGAAAGTGGAACCAAGATTT
CGTCAGAAGAAGAGATCTCAGGAAAGGTGATGAGATCGGATTTCAT
TGGGATCCATATAATTGCGTTTTCAATTTCTGTGTCCTTAAACAAG
CTATGCCAGAGAATTAA
SEQ ID NO: 8 (E1-D13 amino acid sequence; Lof):
MSLQSQSPSKFTTLFISSNMSNPSDEREQCQKKRKSTICEASNFRT
SRATTKMKRR
SEQ ID NO: 9 (E1-D25 CDS sequence; Lof):
ATGAGTTTACAATCTCAAAGCCCATCAAAGTTCACGACCCTATTCA
TCTCTTCCAACATGAGCAACCCTTCAGATGAAAGGGAGCAGTGTCA
AAAGAAGAGGAAATCCACCATATGCGAAGCCTCTAACTCAGCAACA
ACAAAAATGAAGAGGAGATGAACAATAAGGGAGTTTCAACAACACT
GAAGCTTTACGATGATCCTTGGAAGATCAAGAAGACGCTAACCGAT
AGCGATTTGGGAATCCTAAGTAGACTCTTGCTGGCTGCAGATTTGG
TGAAGAAACAAATTTTGCCTATGTTGGGTGCATATCATGCAAGAGC
TGCAGAAACTGAAGGGACCCCAGTTAGAGTTTGGGACATGGACACC
AAATCCATGCACCAACTCGTTCTAAAGCGATGGTCTTCATCCAAGA
GCTATGTTCTTATTGGAAAGTGGAACCAAGATTTCGTCAGAAGAAG
AGATCTCAGGAAAGGTGATGAGATCGGATTTCATTGGGATCCATAT
AATTGCGTTTTCAATTTCTGTGTCCTTAAACAAGCTATGCCAGAGA
ATTAA
SEQ ID NO: 10 (E1-D25 amino acid sequence; Lof):
MSLQSQSPSKFTTLFISSNMSNPSDEREQCQKKRKSTICEASNSAT
TKMKRR
SEQ ID NO: 11 (E1-D9 CDS sequence; Lof):
ATGAGTTTACAATCTCAAAGCCCATCAAAGTTCACGACCCTATTCA
TCTCTTCCAACATGAGCAACCCTTCAGATGAAAGGGAGCAGTGTCA
AAAGAAGAGGAAATCCACCATATGCGAAGCCTCTAACTTTAGGACA
TCAAGGAGCAGCAACAACAAAAATGAAGAGGAGATGAACAATAAGG
GAGTTTCAACAACACTGAAGCTTTACGATGATCCTTGGAAGATCAA
GAAGACGCTAACCGATAGCGATTTGGGAATCCTAAGTAGACTCTTG
CTGGCTGCAGATTTGGTGAAGAAACAAATTTTGCCTATGTTGGGTG
CATATCATGCAAGAGCTGCAGAAACTGAAGGGACCCCAGTTAGAGT
TTGGGACATGGACACCAAATCCATGCACCAACTCGTTCTAAAGCGA
TGGTCTTCATCCAAGAGCTATGTTCTTATTGGAAAGTGGAACCAAG
ATTTCGTCAGAAGAAGAGATCTCAGGAAAGGTGATGAGATCGGATT
TCATTGGGATCCATATAATTGCGTTTTCAATTTCTGTGTCCTTAAA
CAAGCTATGCCAGAGAATTAA
SEQ ID NO: 12 (E1-D9 amino acid sequence; Lof):
MSLQSQSPSKFTTLFISSNMSNPSDEREQCQKKRKSTICEASNFRT
SRSSNNKNEEEMNNKGVSTTLKLYDDPWKIKKTLTDSDLGILSRLL
LAADLVKKQILPMLGAYHARAAETEGTPVRVWDMDTKSMHQLVLKR
WSSSKSYVLIGKWNQDFVRRRDLRKGDEIGFHWDPYNCVFNFCVLK
QAMPEN
SEQ ID NO: 13 (E1-S15 CDS sequence; Lof):
ATGAGTTTACAATCTCAAAGCCCATCAAAGTTCACGACCCTATTCA
TCTCTTCCAACATGAGCAACCCTTCAGATGAAAGGGAGCAGTGTCA
AAAGAAGAGGAAATCCACCATATGCGAAGCCTCTAACTTTAGGACA
TCAAGGAGTTTCAACAACATGAACAACAAAAATGAAGAGGAGATGA
ACAATAAGGGAGTTTCAACAACACTGAAGCTTTACGATGATCCTTG
GAAGATCAAGAAGACGCTAACCGATAGCGATTTGGGAATCCTAAGT
AGACTCTTGCTGGCTGCAGATTTGGTGAAGAAACAAATTTTGCCTA
TGTTGGGTGCATATCATGCAAGAGCTGCAGAAACTGAAGGGACCCC
AGTTAGAGTTTGGGACATGGACACCAAATCCATGCACCAACTCGTT
CTAAAGCGATGGTCTTCATCCAAGAGCTATGTTCTTATTGGAAAGT
GGAACCAAGATTTCGTCAGAAGAAGAGATCTCAGGAAAGGTGATGA
GATCGGATTTCATTGGGATCCATATAATTGCGTTTTCAATTTCTGT
GTCCTTAAACAAGCTATGCCAGAGAATTAA
SEQ ID NO: 14 (E1-S15 amino acid sequence; Lof):
MSLQSQSPSKFTTLFISSNMSNPSDEREQCQKKRKSTICEASNFRT
SRSFNNMNNKNEEEMNNKGVSTTLKLYDDPWKIKKTLTDSDLGILS
RLLLAADLVKKQILPMLGAYHARAAETEGTPVRVWDMDTKSMHQLV
LKRWSSSKSYVLIGKWNQDFVRRRDLRKGDEIGFHWDPYNCVFNFC
VLKQAMPEN
SEQ ID NO: 15 (E1LB-D7 CDS sequence; Lof):
ATGAGTTTACAATCACAAATCCCATCAAAGTTTAAAATAATATTCA
TCTCTTCCAACATGAGCAATCATTCAGATGAGAAGGAGCAGTGTCA
AAAGAAGAGGAAATCCACCATATGTGAAGCCTCCAACTTTAGGACA
TCAAGGAGAACAGCAACAAAAATGAAGAGGAGATGAACAAGGGAGT
TTCAACAACACTGAAGCTTTATGATGACCCTTGGAAGATCAAGAAG
ACGCTAACCGATAGCGATTTGGGAATCCTAAGTAGACTCTCGCTGG
CTACAGATTTGGTGAAGAAGCAAATTTTGCCTATGTTGGGTGCAGA
TCATGCAAGAGCTGCAGAAACTGAAGAAGGGACCCCAGTTAGAGTT
TGGGACATGGACACCAAATCCATGCACCAGCTCGTTCTAAAGCGAT
GGTCTTCTTCCAAGAGCTATGTTCTTATTGCAAAGTGGAACCAAGA
TTTCGTGAGAAGAAGAGACCTCAAGAAAGGGGATGAGATCGGATTT
CATTGGGATCCATATAATTGCGTTTTCAATTTCTGTGTCCTTAAAC
GAGCTATGCCAGAGAATTAA
SEQ ID NO: 16 (E1LB-D7 amino acid sequence; Lof):
MSLQSQIPSKFKIIFISSNMSNHSDEKEQCQKKRKSTICEASNFRT
SRRTATKMKRR
SEQ ID NO: 17 (E1LB-D139 CDS sequence; Lof):
ATGAGTTTACAATCTGCAGCAACAAAAATGAAGAGGAGATGAACAA
GGGAGTTTCAACAACACTGAAGCTTTATGATGACCCTTGGAAGATC
AAGAAGACGCTAACCGATAGCGATTTGGGAATCCTAAGTAGACTCT
CGCTGGCTACAGATTTGGTGAAGAAGCAAATTTTGCCTATGTTGGG
TGCAGATCATGCAAGAGCTGCAGAAACTGAAGAAGGGACCCCAGTT
AGAGTTTGGGACATGGACACCAAATCCATGCACCAGCTCGTTCTAA
AGCGATGGTCTTCTTCCAAGAGCTATGTTCTTATTGCAAAGTGGAA
CCAAGATTTCGTGAGAAGAAGAGACCTCAAGAAAGGGGATGAGATC
GGATTTCATTGGGATCCATATAATTGCGTTTTCAATTTCTGTGTCC
TTAAACGAGCTATGCCAGAGAATTAA
SEQ ID NO: 18 (E1LB-D139 amino acid sequence; Lof):
MSLQSAATKMKRR
SEQ ID NO: 19 (E1LB-D3 CDS sequence; Lof):
ATGAGTTTACAATCACAAATCCCATCAAAGTTTAAAATAATATTCA
TCTCTTCCAACATGAGCAATCATTCAGATGAGAAGGAGCAGTGTCA
AAAGAAGAGGAAATCCACCATATGTGAAGCCTCCAACTTTAGGACA
TCAAGGAGAATCTGCAGCAACAAAAATGAAGAGGAGATGAACAAGG
GAGTTTCAACAACACTGAAGCTTTATGATGACCCTTGGAAGATCAA
GAAGACGCTAACCGATAGCGATTTGGGAATCCTAAGTAGACTCTCG
CTGGCTACAGATTTGGTGAAGAAGCAAATTTTGCCTATGTTGGGTG
CAGATCATGCAAGAGCTGCAGAAACTGAAGAAGGGACCCCAGTTAG
AGTTTGGGACATGGACACCAAATCCATGCACCAGCTCGTTCTAAAG
CGATGGTCTTCTTCCAAGAGCTATGTTCTTATTGCAAAGTGGAACC
AAGATTTCGTGAGAAGAAGAGACCTCAAGAAAGGGGATGAGATCGG
ATTTCATTGGGATCCATATAATTGCGTTTTCAATTTCTGTGTCCTT
AAACGAGCTATGCCAGAGAATTAA
SEQ ID NO: 20 (E1LB-D3 amino acid sequence; Lof):
MSLQSQIPSKFKIIFISSNMSNHSDEKEQCQKKRKSTICEASNFRT
SRRICSNKNEEEMNKGVSTTLKLYDDPWKIKKTLTDSDLGILSRLS
LATDLVKKQILPMLGADHARAAETEEGTPVRVWDMDTKSMHQLVLK
RWSSSKSYVLIAKWNQDFVRRRDLKKGDEIGFHWDPYNCVFNFCVL
KRAMPEN
SEQ ID NO: 21 (E1LB-D6 CDS sequence; Lof):
ATGAGTTTACAATCACAAATCCCATCAAAGTTTAAAATAATATTCA
TCTCTTCCAACATGAGCAATCATTCAGATGAGAAGGAGCAGTGTCA
AAAGAAGAGGAAATCCACCATATGTGAAGCCTCCAACTTTAGGACA
TCAAGGAGAAGCAGCAACAAAAATGAAGAGGAGATGAACAAGGGAG
TTTCAACAACACTGAAGCTTTATGATGACCCTTGGAAGATCAAGAA
GACGCTAACCGATAGCGATTTGGGAATCCTAAGTAGACTCTCGCTG
GCTACAGATTTGGTGAAGAAGCAAATTTTGCCTATGTTGGGTGCAG
ATCATGCAAGAGCTGCAGAAACTGAAGAAGGGACCCCAGTTAGAGT
TTGGGACATGGACACCAAATCCATGCACCAGCTCGTTCTAAAGCGA
TGGTCTTCTTCCAAGAGCTATGTTCTTATTGCAAAGTGGAACCAAG
ATTTCGTGAGAAGAAGAGACCTCAAGAAAGGGGATGAGATCGGATT
TCATTGGGATCCATATAATTGCGTTTTCAATTTCTGTGTCCTTAAA
CGAGCTATGCCAGAGAATTAA
SEQ ID NO: 22 (E1LB-D6 amino acid sequence; Lof):
MSLQSQIPSKFKIIFISSNMSNHSDEKEQCQKKRKSTICEASNFRT
SRRSSNKNEEEMNKGVSTTLKLYDDPWKIKKTLTDSDLGILSRLSL
ATDLVKKQILPMLGADHARAAETEEGTPVRVWDMDTKSMHQLVLKR
WSSSKSYVLIAKWNQDFVRRRDLKKGDEIGFHWDPYNCVFNFCVLK
RAMPEN
SEQ ID NO: 23 (E1LB-D9 CDS sequence; Lof):
ATGAGTTTACAATCACAAATCCCATCAAAGTTTAAAATAATATTCA
TCTCTTCCAACATGAGCAATCATTCAGATGAGAAGGAGCAGTGTCA
AAAGAAGAGGAAATCCACCATATGTGAAGCCTCCAACTTTAGGACA
TCAAGGAGAAGCAACAAAAATGAAGAGGAGATGAACAAGGGAGTTT
CAACAACACTGAAGCTTTATGATGACCCTTGGAAGATCAAGAAGAC
GCTAACCGATAGCGATTTGGGAATCCTAAGTAGACTCTCGCTGGCT
ACAGATTTGGTGAAGAAGCAAATTTTGCCTATGTTGGGTGCAGATC
ATGCAAGAGCTGCAGAAACTGAAGAAGGGACCCCAGTTAGAGTTTG
GGACATGGACACCAAATCCATGCACCAGCTCGTTCTAAAGCGATGG
TCTTCTTCCAAGAGCTATGTTCTTATTGCAAAGTGGAACCAAGATT
TCGTGAGAAGAAGAGACCTCAAGAAAGGGGATGAGATCGGATTTCA
TTGGGATCCATATAATTGCGTTTTCAATTTCTGTGTCCTTAAACGA
GCTATGCCAGAGAATTAA
SEQ ID NO: 24 (E1LB-D9 amino acid sequence; Lof):
MSLQSQIPSKFKIIFISSNMSNHSDEKEQCQKKRKSTICEASNFRT
SRRSNKNEEEMNKGVSTTLKLYDDPWKIKKTLTDSDLGILSRLSLA
TDLVKKQILPMLGADHARAAETEEGTPVRVWDMDTKSMHQLVLKRW
SSSKSYVLIAKWNQDFVRRRDLKKGDEIGFHWDPYNCVFNFCVLKR
AMPEN
SEQ ID NO: 25 (Forward primer for E1Lb):
GTTATCATGCTTGAAAATGATGATG
SEQ ID NO: 26 (Reverse primer for E1Lb):
GTGCATAGATCTCATGCTAGACCT
SEQ ID NO: 27 (Forward primer for E1):
TCCATTGTTTGTAGCGCATGGTC
SEQ ID NO: 28 (Reverse primer for E1):
CATAGATCTCCTGCTAGCTAGACC
SEQ ID NO: 29 (Sequencing primer for E1):
GCTAGTAGTGTAAACACTCAAAACAC
SEQ ID NO: 30 (Sequencing primer for E1Lb):
TGCCTTTCTACTGGCTATGAC
SEQ ID NO: 31 (Forward primer for Cas12a):
GGGCAAGCTCTACATGTTCCAG
SEQ ID NO: 32 (Reverse primer for Cas12a):
CCGTGGTTGTTCTCGTCGA
SEQ ID NO: 33 (Forward primer for Adh):
AGGTGTGGATCGGGCTGTT
SEQ ID NO: 34 (Reverse primer for Adh):
CATCGTGGACGCATTCGA
SEQ ID NO: 35 (Probe for Cas12a):
CGTGGCTCTTGTCGGAGAAGTCCTTGTT
SEQ ID NO: 36 (Probe for Adh):
ACTGGCAGCATCCAAGCCATGGTCT
SEQ ID NO: 37 (gRNA used for targeting
second NLS basic domain of E1 and E1Lb
loci) (PAM: TTTA):
TTTAGGACATCAAGGAGAAGATTCTGC

1. A method of establishing where a soybean plant, or seed thereof, should be grown, comprising:
a. introducing, via genome modification using a site directed nuclease, a mutation at a nuclear localization signal (NLS) of an E1 locus and/or an E1 LB locus of a genome of a soybean plant;
b. selfing the plant for one or more generations to generate a progeny plant that is homozygous for the mutation at said E1 locus and/or said E1 LB locus;
c. obtaining DNA from said progeny plant;
d. determining an allelic combination of said progeny plant via an assay indicative of the mutation introduced at said E1 locus and/or said E1 LB locus; and
e. assigning a change in flowering time and/or maturity time to the progeny plant based on the determined allelic combination, wherein the change in flowering time and/or maturity time is relative to a control plant not comprising the determined allelic combination.

2. (canceled)

3. (canceled)

4. (canceled)

5. The method of claim 1, wherein the introducing comprises transforming a plant cell with an expression cassette comprising:
(i) a nucleic acid that encodes the site-directed nuclease; and
(ii) a nucleic acid that encodes at least one guide RNA (gRNA) directed to a target sequence comprising a second basic domain of the NLS at said E1 locus and/or said E1 LB locus.

6. (canceled)

7. (canceled)

8. The method of claim 5, wherein the nucleic acid that encodes the site-directed nuclease is operably linked to a first promoter of the expression cassette and wherein the nucleic acid that encodes the at least one gRNA is operably linked to a second promoter of the expression cassette and, optionally, wherein the expression cassette further comprises an enhancer operably linked to the first promoter or the second promoter.

9. (canceled)

10. (canceled)

11. (canceled)

12. The method of claim 1, wherein the allelic combination comprises one or more of:
a) a mutant E1 allele resulting in a loss of function of an E1 protein;
b) a mutant E1 allele resulting in partial function of the E1 protein;
c) a mutant E1 LB allele resulting in a loss of function of an E1 LB protein; and
d) a mutant E1 LB allele resulting in partial function of the E1 LB protein.

13. The method of claim 1, wherein the mutant allele at the E1 LB locus corresponds to any one of SEQ ID NOS: 15, 17, 19, 21 and 23; or encodes a mutant E1 LB protein corresponding to any one of SEQ ID NOS: 16, 18, 20, 22 and 24; and wherein the mutant allele at the E1 locus corresponds to any one of SEQ ID NOS: 5, 7, 9, 11 and 13; or encodes a mutant E1 protein corresponding to any one of SEQ ID NOS: 6, 8, 10, 12 and 14.

14. The method of claim 1, wherein the progeny plant has a modified flowering time and/or maturity time relative to the control plant, and wherein the control plant comprises a wild-type allele at each of the E1 locus and E1 LB locus.

15. The method of claim 1, wherein assigning a changing in flowering time and/or maturity time comprises assigning a number of days by which the flowering time and/or maturity time is shortened for the progeny plant relative to the control plant.

16. (canceled)

17. The method of claim 5, wherein the introducing further comprises:
regenerating a transformed T0 plant from the transformed plant cell, the transformed T0 plant having a plurality of T1 seed, wherein the plurality of T1 seed contain a plurality of unique edits in the E1 locus and/or the E1 LB locus;
growing a plurality of T1 plants from the T1 seed; and
selfing the T1 plants for one or more generations to obtain a progeny plant that is homozygous for the introduced mutation at the E1 locus and/or the E1 LB locus.

18. (canceled)

19. A method of producing a soybean plant with a modified flowering time, comprising:
introducing an edit into an NLS of one or more of an E1 gene and an E1 LB gene of a soybean plant cell to generate a mutant E1 allele and/or a mutant E1 LB allele having reduced function relative to a corresponding wild-type allele;
regenerating an edited plant from the edited plant cell; and
selfing the edited plant to obtain an edited progeny plant having a non-naturally occurring allelic combination comprising one or more of the mutant E1 allele and the mutant E1 LB allele, wherein the edited progeny plant has a flowering time and/or maturity time that is modified relative to the flowering time and/or maturity time of a control plant comprising the wild-type E1 allele and the wild-type E1 LB allele.

20. The method of claim 19, wherein introducing the edit into the E1 gene comprises introducing a plurality of base pair deletions into the E1 gene, wherein the mutant E1 allele corresponds to any one of SEQ ID NOS: 5, 7, 9, 11 and 13 and encodes an in-frame deletion mutant E1 protein or a truncated E1 protein corresponding to any one of SEQ ID NOS: 6, 8, 10, 12 and 14; and wherein introducing the edit into the E1 LB gene comprises introducing a plurality of base pair deletions into the E1 LB gene, wherein the mutant E1 LB allele corresponds to any one of SEQ ID NOS: 15, 17, 19, 21 and 23 and encodes an in-frame deletion mutant E1 LB protein or a truncated E1 LB protein corresponding to any one of SEQ ID NOS: 16, 18, 20, 22 and 24, wherein the truncated E1 protein has no functional activity relative to a wild-type E1 protein, wherein the in-frame deletion mutant E1 protein has partial functional activity relative to the wild-type E1 protein, wherein the truncated E1 LB protein has no functional activity relative to a wild-type E1 LB protein, and wherein the in-frame deletion mutant E1 LB protein has partial functional activity relative to the wild-type E1 LB protein.

21. The method of claim 19, wherein introducing the edit into the E1 and/or E1 LB gene comprises introducing the edit into a second basic domain of a nuclear localization signal (NLS) of the E1 gene, the E1 LB gene, or both by transforming the plant cell with an expression cassette comprising: (i) a nucleic acid that encodes a site-directed nuclease operably linked to a promoter, the promoter optionally further linked to an enhancer configured to enhance transcription of the site directed nuclease by the promoter; and (ii) a nucleic acid that encodes at least one guide RNA (gRNA) directed to a target sequence in the second basic domain of the NLS of the E1 gene and/or the E1 LB gene.

22. (canceled)

23. The method of claim 19, wherein the flowering time of the edited progeny plant is shorter than the flowering time of the control plant and/or wherein a relative maturity group value of the edited progeny plant is different from the relative maturity group value of the control plant.

24. (canceled)

25. The edited progeny plant of the method of claim 19.

26. A further progeny plant of the edited progeny plant of claim 25, wherein the further progeny plant is obtained by breeding or selfing.

27. A non-naturally occurring soybean plant having a modified flowering time, comprising an allele combination selected from the group comprising:
(a) a mutant E1 LB allele resulting in partial expression of E1 LB protein and a wild-type E1 allele;
(b) a mutant E1 LB allele resulting in loss of expression of E1 LB protein and a wild-type E1 allele;
(c) a mutant E1 LB allele resulting in partial expression of E1 LB protein and a mutant E1 allele resulting in partial expression of E1 protein;
(d) a mutant E1 LB allele resulting in loss of expression of E1 LB protein and a mutant E1 allele resulting in partial expression of E1 protein;
(e) a mutant E1 LB allele resulting in partial expression of E1 LB protein and a mutant E1 allele resulting in loss of expression of E1 protein;
(f) a mutant E1 LB allele resulting in loss of expression of E1 LB protein and a mutant E1 allele resulting in loss of expression of E1 protein;
(g) a mutant E1 allele resulting in partial expression of E1 protein and a wild-type E1 LB allele; and
(h) a mutant E1 allele resulting in loss of expression of E1 protein and a wild-type E1 LB allele; wherein the flowering time of the non-naturally occurring soybean plant is modified relative to a control plant comprising the wild-type E1 allele and a wild-type E1 LB allele.

28. The soybean plant of claim 27, wherein the mutant E1 LB allele corresponds to any one of SEQ ID NOS: 15, 17, 19, 21 and 23; or encodes a mutant E1 LB protein corresponding to any one of SEQ ID NOS: 16, 18, 20, 22 and 24; and wherein the mutant E1 allele corresponds to any one of SEQ ID NOS: 5, 7, 9, 11 and 13; or encodes a mutant E1 protein corresponding to any one of SEQ ID NOS: 6, 8, 10, 12 and 14.

29. The soybean plant of claim 27, wherein the mutant E1 LB allele and/or the mutant E1 LB allele are introduced by editing the respective gene of the plant using a DNA modification enzyme.

30. (canceled)

31. A method of breeding, comprising:
crossing the non-naturally occurring soybean plant of claim 27 with a different soybean plant not comprising the allele combination of the non-naturally occurring soybean plant; and
selecting a progeny plant having the modified flowering.

32. A modified soybean plant, or plant part thereof, comprising a non-naturally occurring mutant allele at an E1 locus and/or a E1 Lb locus, wherein the non-naturally occurring mutant allele is introduced via genome modification using a site directed nuclease.

33. (canceled)

34. (canceled)

35. (canceled)

36. (canceled)

37. The modified soybean plant, or plant part thereof, of claim 32, wherein said mutant allele at the E1 locus and/or the E1 LB locus comprises one or more mutation types selected from the group consisting of a nonsense mutation, an in-frame deletion mutation, a missense mutation, a frameshift mutation, a splice-site mutation, and any combination thereof.

38. (canceled)

39. The modified soybean plant, or plant part thereof, of claim 32, wherein said modified plant has a smaller flowering time or a smaller maturity time, or both, than that of a control plant comprising an unmodified wild-type E1 and/or wild-type E1 LB gene allele.

40. (canceled)

41. (canceled)

42. (canceled)

43. The modified soybean plant, or plant part thereof, of claim 32, wherein said homozygous mutant allele comprises a homozygous mutation at the E1 locus selected from the group consisting of:
(a) deletion of 8 bp from position 147 to 154 of the E1 gene;
(b) deletion of 13 bp from position 144 to 156 of the E1 gene;
(c) deletion of 25 bp from position 131 to 155 of the E1 gene;
(d) deletion of 9 bp from position 146 to 154 of the E1 gene; and
(e) substitution of 15 bp from position 145 to 159 of the E1 gene,
wherein said position is with reference to the E1 gene of SEQ ID NO. 1.

44. (canceled)

45. The modified soybean plant, or plant part thereof, of claim 32, wherein said homozygous mutant allele comprises a homozygous mutation at the E1 LB locus selected from the group consisting of:
(a) deletion of 7 bp from position 149 to 155 of the E1 LB gene;
(b) deletion of 3 bp from position 149 to 151 of the E1 LB gene;
(c) deletion of 139 bp from position 13 to 151 of the E1 LB gene;
(d) deletion of 6 bp from position 149 to 154 of the E1 LB gene; and
(e) deletion of 9 bp from position 148 to 156 of the E1 LB gene,
wherein said position is with reference to the E1 LB gene of SEQ ID NO. 3.

46. (canceled)


SEQUENCE LISTING (37 sequences)

SEQ ID NO: 1 - DNA, 582 bp, Glycine max
  source 1..582: mol_type = other DNA
atgagtttacaatctcaaagcccatcaaagttcacgaccctattcatctcttccaacatgagcaacccttcagatgaaagggagcagtgtcaaaagaagaggaaatccaccatatgcgaagcctctaactttaggacatcaaggagaagattctgcagcaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaa

SEQ ID NO: 2 - protein, 193 aa, Glycine max
MSLQSQSPSKFTTLFISSNMSNPSDEREQCQKKRKSTICEASNFRTSRRRFCSNNKNEEEMNNKGVSTTLKLYDDPWKIKKTLTDSDLGILSRLLLAADLVKKQILPMLGAYHARAAETEGTPVRVWDMDTKSMHQLVLKRWSSSKSYVLIGKWNQDFVRRRDLRKGDEIGFHWDPYNCVFNFCVLKQAMPEN

SEQ ID NO: 3 - DNA, 579 bp, Glycine max
  source 1..579: mol_type = other DNA
atgagtttacaatcacaaatcccatcaaagtttaaaataatattcatctcttccaacatgagcaatcattcagatgagaaggagcagtgtcaaaagaagaggaaatccaccatatgtgaagcctccaactttaggacatcaaggagaagattctgcagcaacaaaaatgaagaggagatgaacaagggagtttcaacaacactgaagctttatgatgacccttggaagatcaagaagacgctaaccgatagcgatttgggaatcctaagtagactctcgctggctacagatttggtgaagaagcaaattttgcctatgttgggtgcagatcatgcaagagctgcagaaactgaagaagggaccccagttagagtttgggacatggacaccaaatccatgcaccagctcgttctaaagcgatggtcttcttccaagagctatgttcttattgcaaagtggaaccaagatttcgtgagaagaagagacctcaagaaaggggatgagatcggatttcattgggatccatataattgcgttttcaatttctgtgtccttaaacgagctatgccagagaattaa

SEQ ID NO: 4 - protein, 192 aa, Glycine max
MSLQSQIPSKFKIIFISSNMSNHSDEKEQCQKKRKSTICEASNFRTSRRRFCSNKNEEEMNKGVSTTLKLYDDPWKIKKTLTDSDLGILSRLSLATDLVKKQILPMLGADHARAAETEEGTPVRVWDMDTKSMHQLVLKRWSSSKSYVLIAKWNQDFVRRRDLKKGDEIGFHWDPYNCVFNFCVLKRAMPEN

SEQ ID NO: 5 - DNA, 574 bp, Glycine max
  source 1..574: mol_type = other DNA
atgagtttacaatctcaaagcccatcaaagttcacgaccctattcatctcttccaacatgagcaacccttcagatgaaagggagcagtgtcaaaagaagaggaaatccaccatatgcgaagcctctaactttaggacatcaaggaggcagcaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaa

SEQ ID NO: 6 - protein, 54 aa, Glycine max
MSLQSQSPSKFTTLFISSNMSNPSDEREQCQKKRKSTICEASNFRTSRRQQQQK

SEQ ID NO: 7 - DNA, 569 bp, Glycine max
  source 1..569: mol_type = other DNA
atgagtttacaatctcaaagcccatcaaagttcacgaccctattcatctcttccaacatgagcaacccttcagatgaaagggagcagtgtcaaaagaagaggaaatccaccatatgcgaagcctctaactttaggacatcaagagcaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaa

SEQ ID NO: 8 - protein, 56 aa, Glycine max
MSLQSQSPSKFTTLFISSNMSNPSDEREQCQKKRKSTICEASNFRTSRATTKMKRR

SEQ ID NO: 9 - DNA, 557 bp, Glycine max
  source 1..557: mol_type = other DNA
atgagtttacaatctcaaagcccatcaaagttcacgaccctattcatctcttccaacatgagcaacccttcagatgaaagggagcagtgtcaaaagaagaggaaatccaccatatgcgaagcctctaactcagcaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaa

SEQ ID NO: 10 - protein, 52 aa, Glycine max
MSLQSQSPSKFTTLFISSNMSNPSDEREQCQKKRKSTICEASNSATTKMKRR

SEQ ID NO: 11 - DNA, 573 bp, Glycine max
  source 1..573: mol_type = other DNA
atgagtttacaatctcaaagcccatcaaagttcacgaccctattcatctcttccaacatgagcaacccttcagatgaaagggagcagtgtcaaaagaagaggaaatccaccatatgcgaagcctctaactttaggacatcaaggagcagcaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaa

SEQ ID NO: 12 - protein, 190 aa, Glycine max
MSLQSQSPSKFTTLFISSNMSNPSDEREQCQKKRKSTICEASNFRTSRSSNNKNEEEMNNKGVSTTLKLYDDPWKIKKTLTDSDLGILSRLLLAADLVKKQILPMLGAYHARAAETEGTPVRVWDMDTKSMHQLVLKRWSSSKSYVLIGKWNQDFVRRRDLRKGDEIGFHWDPYNCVFNFCVLKQAMPEN

SEQ ID NO: 13 - DNA, 582 bp, Glycine max
  source 1..582: mol_type = other DNA
atgagtttacaatctcaaagcccatcaaagttcacgaccctattcatctcttccaacatgagcaacccttcagatgaaagggagcagtgtcaaaagaagaggaaatccaccatatgcgaagcctctaactttaggacatcaaggagtttcaacaacatgaacaacaaaaatgaagaggagatgaacaataagggagtttcaacaacactgaagctttacgatgatccttggaagatcaagaagacgctaaccgatagcgatttgggaatcctaagtagactcttgctggctgcagatttggtgaagaaacaaattttgcctatgttgggtgcatatcatgcaagagctgcagaaactgaagggaccccagttagagtttgggacatggacaccaaatccatgcaccaactcgttctaaagcgatggtcttcatccaagagctatgttcttattggaaagtggaaccaagatttcgtcagaagaagagatctcaggaaaggtgatgagatcggatttcattgggatccatataattgcgttttcaatttctgtgtccttaaacaagctatgccagagaattaa

SEQ ID NO: 14 - protein, 193 aa, Glycine max
MSLQSQSPSKFTTLFISSNMSNPSDEREQCQKKRKSTICEASNFRTSRSFNNMNNKNEEEMNNKGVSTTLKLYDDPWKIKKTLTDSDLGILSRLLLAADLVKKQILPMLGAYHARAAETEGTPVRVWDMDTKSMHQLVLKRWSSSKSYVLIGKWNQDFVRRRDLRKGDEIGFHWDPYNCVFNFCVLKQAMPEN

SEQ ID NO: 15 - DNA, 572 bp, Glycine max
  source 1..572: mol_type = other DNA
atgagtttacaatcacaaatcccatcaaagtttaaaataatattcatctcttccaacatgagcaatcattcagatgagaaggagcagtgtcaaaagaagaggaaatccaccatatgtgaagcctccaactttaggacatcaaggagaacagcaacaaaaatgaagaggagatgaacaagggagtttcaacaacactgaagctttatgatgacccttggaagatcaagaagacgctaaccgatagcgatttgggaatcctaagtagactctcgctggctacagatttggtgaagaagcaaattttgcctatgttgggtgcagatcatgcaagagctgcagaaactgaagaagggaccccagttagagtttgggacatggacaccaaatccatgcaccagctcgttctaaagcgatggtcttcttccaagagctatgttcttattgcaaagtggaaccaagatttcgtgagaagaagagacctcaagaaaggggatgagatcggatttcattgggatccatataattgcgttttcaatttctgtgtccttaaacgagctatgccagagaattaa

SEQ ID NO: 16 - protein, 57 aa, Glycine max
MSLQSQIPSKFKIIFISSNMSNHSDEKEQCQKKRKSTICEASNFRTSRRTATKMKRR

SEQ ID NO: 17 - DNA, 440 bp, Glycine max
  source 1..440: mol_type = other DNA
atgagtttacaatctgcagcaacaaaaatgaagaggagatgaacaagggagtttcaacaacactgaagctttatgatgacccttggaagatcaagaagacgctaaccgatagcgatttgggaatcctaagtagactctcgctggctacagatttggtgaagaagcaaattttgcctatgttgggtgcagatcatgcaagagctgcagaaactgaagaagggaccccagttagagtttgggacatggacaccaaatccatgcaccagctcgttctaaagcgatggtcttcttccaagagctatgttcttattgcaaagtggaaccaagatttcgtgagaagaagagacctcaagaaaggggatgagatcggatttcattgggatccatataattgcgttttcaatttctgtgtccttaaacgagctatgccagagaattaa

SEQ ID NO: 18 - protein, 13 aa, Glycine max
MSLQSAATKMKRR

SEQ ID NO: 19 - DNA, 576 bp, Glycine max
  source 1..576: mol_type = other DNA
atgagtttacaatcacaaatcccatcaaagtttaaaataatattcatctcttccaacatgagcaatcattcagatgagaaggagcagtgtcaaaagaagaggaaatccaccatatgtgaagcctccaactttaggacatcaaggagaatctgcagcaacaaaaatgaagaggagatgaacaagggagtttcaacaacactgaagctttatgatgacccttggaagatcaagaagacgctaaccgatagcgatttgggaatcctaagtagactctcgctggctacagatttggtgaagaagcaaattttgcctatgttgggtgcagatcatgcaagagctgcagaaactgaagaagggaccccagttagagtttgggacatggacaccaaatccatgcaccagctcgttctaaagcgatggtcttcttccaagagctatgttcttattgcaaagtggaaccaagatttcgtgagaagaagagacctcaagaaaggggatgagatcggatttcattgggatccatataattgcgttttcaatttctgtgtccttaaacgagctatgccagagaattaa

SEQ ID NO: 20 - protein, 191 aa, Glycine max
MSLQSQIPSKFKIIFISSNMSNHSDEKEQCQKKRKSTICEASNFRTSRRICSNKNEEEMNKGVSTTLKLYDDPWKIKKTLTDSDLGILSRLSLATDLVKKQILPMLGADHARAAETEEGTPVRVWDMDTKSMHQLVLKRWSSSKSYVLIAKWNQDFVRRRDLKKGDEIGFHWDPYNCVFNFCVLKRAMPEN

SEQ ID NO: 21 - DNA, 573 bp, Glycine max
  source 1..573: mol_type = other DNA
atgagtttacaatcacaaatcccatcaaagtttaaaataatattcatctcttccaacatgagcaatcattcagatgagaaggagcagtgtcaaaagaagaggaaatccaccatatgtgaagcctccaactttaggacatcaaggagaagcagcaacaaaaatgaagaggagatgaacaagggagtttcaacaacactgaagctttatgatgacccttggaagatcaagaagacgctaaccgatagcgatttgggaatcctaagtagactctcgctggctacagatttggtgaagaagcaaattttgcctatgttgggtgcagatcatgcaagagctgcagaaactgaagaagggaccccagttagagtttgggacatggacaccaaatccatgcaccagctcgttctaaagcgatggtcttcttccaagagctatgttcttattgcaaagtggaaccaagatttcgtgagaagaagagacctcaagaaaggggatgagatcggatttcattgggatccatataattgcgttttcaatttctgtgtccttaaacgagctatgccagagaattaa

SEQ ID NO: 22 - protein, 190 aa, Glycine max
MSLQSQIPSKFKIIFISSNMSNHSDEKEQCQKKRKSTICEASNFRTSRRSSNKNEEEMNKGVSTTLKLYDDPWKIKKTLTDSDLGILSRLSLATDLVKKQILPMLGADHARAAETEEGTPVRVWDMDTKSMHQLVLKRWSSSKSYVLIAKWNQDFVRRRDLKKGDEIGFHWDPYNCVFNFCVLKRAMPEN

SEQ ID NO: 23 - DNA, 570 bp, Glycine max
  source 1..570: mol_type = other DNA
atgagtttacaatcacaaatcccatcaaagtttaaaataatattcatctcttccaacatgagcaatcattcagatgagaaggagcagtgtcaaaagaagaggaaatccaccatatgtgaagcctccaactttaggacatcaaggagaagcaacaaaaatgaagaggagatgaacaagggagtttcaacaacactgaagctttatgatgacccttggaagatcaagaagacgctaaccgatagcgatttgggaatcctaagtagactctcgctggctacagatttggtgaagaagcaaattttgcctatgttgggtgcagatcatgcaagagctgcagaaactgaagaagggaccccagttagagtttgggacatggacaccaaatccatgcaccagctcgttctaaagcgatggtcttcttccaagagctatgttcttattgcaaagtggaaccaagatttcgtgagaagaagagacctcaagaaaggggatgagatcggatttcattgggatccatataattgcgttttcaatttctgtgtccttaaacgagctatgccagagaattaa

SEQ ID NO: 24 - protein, 189 aa, Glycine max
MSLQSQIPSKFKIIFISSNMSNHSDEKEQCQKKRKSTICEASNFRTSRRSNKNEEEMNKGVSTTLKLYDDPWKIKKTLTDSDLGILSRLSLATDLVKKQILPMLGADHARAAETEEGTPVRVWDMDTKSMHQLVLKRWSSSKSYVLIAKWNQDFVRRRDLKKGDEIGFHWDPYNCVFNFCVLKRAMPEN

SEQ ID NO: 25 - DNA, 25 bp, Glycine max
  source 1..25: mol_type = other DNA
gttatcatgcttgaaaatgatgatg

SEQ ID NO: 26 - DNA, 24 bp, Glycine max
  source 1..24: mol_type = other DNA
gtgcatagatctcatgctagacct

SEQ ID NO: 27 - DNA, 23 bp, Glycine max
  source 1..23: mol_type = other DNA
tccattgtttgtagcgcatggtc

SEQ ID NO: 28 - DNA, 24 bp, Glycine max
  source 1..24: mol_type = other DNA
catagatctcctgctagctagacc

SEQ ID NO: 29 - DNA, 26 bp, Glycine max
  source 1..26: mol_type = other DNA
gctagtagtgtaaacactcaaaacac

SEQ ID NO: 30 - DNA, 21 bp, Glycine max
  source 1..21: mol_type = other DNA
tgcctttctactggctatgac

SEQ ID NO: 31 - DNA, 22 bp, Glycine max
  source 1..22: mol_type = other DNA
gggcaagctctacatgttccag

SEQ ID NO: 32 - DNA, 19 bp, Glycine max
  source 1..19: mol_type = other DNA
ccgtggttgttctcgtcga

SEQ ID NO: 33 - DNA, 19 bp, Glycine max
  source 1..19: mol_type = other DNA
aggtgtggatcgggctgtt

SEQ ID NO: 34 - DNA, 18 bp, Glycine max
  source 1..18: mol_type = other DNA
catcgtggacgcattcga

SEQ ID NO: 35 - DNA, 28 bp, Glycine max
  source 1..28: mol_type = other DNA
cgtggctcttgtcggagaagtccttgtt

SEQ ID NO: 36 - DNA, 25 bp, Glycine max
  source 1..25: mol_type = other DNA
actggcagcatccaagccatggtct

SEQ ID NO: 37 - DNA, 27 bp, Glycine max
  source 1..27: mol_type = other DNA
tttaggacatcaaggagaagattctgc